SEQUENCE LISTING (2 sequences)

SEQ ID NO: 1 - DNA, 50 bp, Candida tropicalis
  misc_feature 1..50: Example 21 oligonucleotide having the sequence of nucleotides 1180-1229 of the C. tropicalis cytochrome P450alk3 gene (Genbank Accession No. Z13010
ccattattaggtgatggtatttttactttggatggtgaaggttggaaaca

SEQ ID NO: 2 - DNA, 20 bp, Artificial Sequence
  misc_feature 1..20: Example 22 target oligonucleotide
tctgagggactttcctgatc

(19) United States
(12) Patent Application Publication
(10) Pub. No.: US 2005/0147994 A1
(43) Pub. Date: Jul. 7, 2005

(54) Method for immobilizing a biologic in a polyurethane-hydrogel composition, a composition prepared from the method, and biomedical applicaions

(76) Inventors: Andrew F. Sandford, Marlborough, MA (US); Venkiteswaran Subramanian, San Diego, CA (US); Jeffrey R. Allen, Poway, CA (US); Paul E. Swanson, San Diego, CA (US); Joey W. Storer, Midland, MI (US); Deepak K. Sharma, Midland, MI (US)

Correspondence Address: THE DOW CHEMICAL COMPANY;INTELLECTUAL PROPERTY SECTION, P. O. BOX 1967, MIDLAND, MI, 48641-1967, US

(21) Appl. No.: 10/979,012

(22) Filed: Nov. 1, 2004

Related U.S. Application Data
(63) Continuation of application No. 10/153,131, filed on May 21, 2002.
(60) Provisional application No. 60/337,797, filed on Dec. 5, 2001.

(51) Int. Cl. C12Q 1/68; G01N 33/53

(52) U.S. Cl. 435/6; 435/7.1; 525/54.1

(57) ABSTRACT

A biologic can be immobilized in a transparent polyurethane-hydrogel composition. Such a polyurethane-hydrogel composition can be prepared from forming a polyurethane-hydrogel mixture and immobilizing a biologic in the mixture. The mixture can be formed by admixing a prepolymer and a water-soluble crosslinker in aqueous solvent and in the substantial absence of organic solvent. A suitable prepolymer generally includes at least one water-soluble polyol and at least one isocyanate and can be added to the mixture in an amount of no greater than about 5 weight percent based on total weight of all components. A suitable crosslinker generally has a crosslinker functionality of at least 2. A biologic includes cells, peptides, nucleics, peptide nucleic acids, saccharides, lipopolysaccharides, glycolipids, and combinations of these. A polyurethane-hydrogel composition having an immobilized biologic can be particularly useful for biomedical applications. Some examples of useful biomedical applications include protein microarrays, cell microarrays, and DNA microarrays.

This application is a continuation of copending application Ser. No. 10/153,131 filed May 21, 2002, now abandoned, which claims the benefit of U.S. Provisional Application No. 60/337,797, filed Dec. 5, 2001.

### FIELD OF THE INVENTION

The invention is directed to a method of immobilizing a biologic in a polyurethane-hydrogel composition and to a composition prepared from this method. More particularly, the invention is directed to preparing a polyurethane-hydrogel composition in the substantial absence of organic solvent. A method of the invention and a composition of the invention are particularly useful for biomedical applications, such as assays useful for diagnostic devices and therapeutic applications.

### BACKGROUND OF THE INVENTION

The analysis of biospecific agents (e.g., small molecules; proteins; and ligands) that selectively interact with biomolecules, such as by catalysis, binding, proteolysis, or other biological interactions, is of particular interest in medicinal chemistry. Such an analysis can be used for diagnostic and therapeutic applications as well as for biomolecule characterization, screening for biological activity, and other functional studies.

Arrays of biomolecules, such as arrays of peptides or arrays of polynucleotides are useful for this type of analysis. Such arrays include regions (sometimes referred to as spots) of usually different sequence biomolecules arranged in a predetermined configuration on a substrate. The arrays, when exposed to a sample, will exhibit a pattern of binding or activity that is indicative of the presence and/or concentration of one or more components of the sample, such as an antigen in the case of a peptide array or a polynucleotide having a particular sequence in the case of a polynucleotide array. The binding pattern can be detected by, for example, labeling all potential targets (e.g., DNA) in the sample with a suitable label (e.g., a fluorescent compound), and observing a signal pattern (e.g., fluorescence) on the array.

Such an analysis generally involves immobilizing a biomolecule on a substrate composition in a manner that preserves the biological activity of the biomolecule. Although a variety of techniques is known to be useful for genetic analysis (e.g., analysis of DNA, RNA, and peptide nucleic acids (PNA)), techniques that are useful for analysis of other water-soluble biomolecules, particularly proteins and peptides, are still needed.

One type of substrate composition that has been used is a polyacrylamide gel. Polyacrylamide gels are less than desirable for some applications because they can be expensive to manufacture and because some reagents used to make polyacrylamide gels can substantially adversely affect some biomolecules. Moreover, the brittle nature of polyacrylamide gels may limit their use in high-throughput applications.

Another type of substrate composition that has been used is a polyurethane gel. A polyurethane gel is created from a polyurethane network and a solvent. The polyurethane network envelopes the solvent and can prevent the solvent from flowing out of the network. The properties of a polyurethane gel depend largely on the structure of the polyurethane network that makes up the gel and the interaction of the network and the solvent. The polyurethane network depends on the crosslink structure of the network, which depends on, for example, the amount and type of the reactants used to make the network and their ability to react to near completion. The polyurethane network can be important for determining the strength of the gel and can also be important for the diffusion of molecules through the gel.

A variety of polyurethane gels is known, and one advantage of some of these gels is that they are transparent. But it can be difficult to formulate a transparent polyurethane gel suitable for analysis of biomolecules. Transparency is determined by the polyurethane network in combination with the solvent as well as the residual reactants. Thus, some reactants that may provide a desirable polyurethane network may be unable to provide transparency, and some reactants that can provide transparency may be unable to provide a desirable network.

The known transparent polyurethane gels are less than desirable because they generally require large amounts of polymer—e.g., more than 5 weight percent and even more than 20 weight percent in some applications. Using such large amounts of polymer can be expensive and can negate or reduce transparency unless large amounts of organic solvent are used to facilitate formation of transparent gel.

Attempts to reduce the amount of polymer in known formulations to no more than 5 weight percent can adversely affect gel formation. And attempts to modify known formulations by altering the reactants such that less than 5 weight percent of polymer can form a desirable gel can adversely affect gel transparency.

At least one polyurethane gel is known to be useful for immobilizing robust biomolecules (PNA, DNA, and RNA). But such a gel is also prepared in an organic solvent, which can be at least partly removed in a washing step after the gel is formed. This washing step can be slow and expensive in high-volume manufacturing applications. Moreover, such conditions are typically too harsh for many biomolecules. That is, water-soluble biomolecules are likely to lose biological activity or to have substantially reduced biological activity when exposed to an organic solvent. For analysis of biomolecules to be useful, it is important to sufficiently maintain the biological activity of the biomolecule to detect the selective interaction between a biomolecule and a biospecific agent.

For example, for a protein to remain biologically active, any formulation to which the protein is exposed must substantially preserve intact the conformational integrity of at least a core sequence of the protein's amino acids while at the same time protecting the protein's multiple functional groups from degradation. Degradation pathways for proteins can involve chemical instability (i.e., any process that involves modification of the protein by bond formation or cleavage resulting in a new chemical entity) or physical instability (i.e., changes in higher order structure of the protein). Chemical instability can result from, for example, deamidation, racemization, hydrolysis, oxidation, reduction, beta elimination, or disulfide exchange. Physical instability can result from, for example, denaturation, aggregation, precipitation, or adsorption. Thus, when immobilizing a protein in a formulation, the formulation conditions need to be mild enough to sufficiently retard chemical instability and physical instability of the protein such that the protein will maintain detectable biological activity.

It would be desirable to immobilize a biomolecule such as a protein or peptide in a polyurethane gel. Moreover, it would be desirable to prepare the gel with no more than 5 weight percent of polymer and to prepare the gel in the substantial absence of volatile organic solvent, while still substantially maintaining the gel's transparency.

### SUMMARY OF THE INVENTION

A polyurethane-hydrogel composition having an immobilized biologic can be prepared by a method including forming a polyurethane-hydrogel mixture and immobilizing a biologic in the mixture. The mixture can be formed by admixing at least one prepolymer and at least one water-soluble crosslinker in aqueous solvent and in the substantial absence of organic solvent. The prepolymer generally is prepared from at least one water-soluble polyol and at least one isocyanate. The crosslinker generally has a crosslinker functionality of at least 2.

In one embodiment, the biologic is immobilized in the composition by derivatizing at least one of the prepolymer and the water-soluble crosslinker with the biologic before admixing the prepolymer and the crosslinker.

In another embodiment, the biologic is immobilized in the composition by admixing the prepolymer, the biologic, and the crosslinker.

In yet another embodiment, the biologic is immobilized in the composition by contacting a polymerized mixture with the biologic.

In one embodiment, a biologic is immobilized in a composition of the invention by use of an immobilizing agent.

Suitable water-soluble crosslinkers include polyethylenimine and an amine end-capped poly(ethylene oxide) crosslinker (e.g., 3-arm amine end-capped polyethyleneglycol). A particularly useful crosslinker according to the invention includes water-soluble crosslinkers selected to optimize nonspecific binding to a composition of the invention. Additional or alternative methods to optimize nonspecific binding include treating a composition of the invention or at least one hydrogel component with a blocking agent.

Other suitable water-soluble crosslinkers include crosslinkers that have a functionality effective to provide a reaction rate with the prepolymer that is at least 10 times faster than the reaction rate of water with the prepolymer.

In one embodiment, a prepolymer is prepared from isophorone diisocyanate and a polyol having a 7,000 molecular-weight triol copolymer of 75% ethylene oxide and 25% propylene oxide.

Suitable biologics include cells, nucleics, peptides, peptide nucleic acids, and saccharides. Particularly preferred biologics include cells and peptides.

A composition of the invention is transparent when substantially polymerized and has a desirable physical property, particularly an effective number-average molecular weight between crosslinks, when polymerized.

A composition of the invention can be particularly useful for biomedical applications, such as assays useful for diagnostic devices and therapeutic applications. Exemplary biomedical applications include cell microarrays, protein microarrays, and DNA microarrays.

A composition of the invention can be included in a biomedical device and can be included in a kit having such a biomedical device and at least one reagent useful for conducting an assay on such a device.

A composition of the invention can be prepared by admixing at least one prepolymer and at least one water-soluble crosslinker in aqueous solvent and in the substantial absence of organic solvent to form a mixture and by contacting the mixture with a biologic.

### BRIEF DESCRIPTION OF THE FIGURES

FIG. 1 illustrates the reaction scheme for lactate dehydrogenase (LDH) catalyzing the oxidation of lactate to pyruvate with concomitant reduction of nicotinamide adenine dinucleotide to NADH.

FIG. 2 illustrates the activity of free lactate dehydrogenase in water (A) and in buffer (C) and the activity of lactate dehydrogenase immobilized in a composition of the invention prepared in water (B) and prepared in buffer (D).

FIG. 3 illustrates the activity of lactate dehydrogenase over time. The samples tested include free lactate dehydrogenase in buffer (B) and lactate dehydrogenase immobilized in a composition of the invention prepared in water (A), in buffer (C), and in buffer supplemented with glycerol (D).

FIG. 4 illustrates another example of the relative activity of lactate dehydrogenase over time. All samples tested included an enzyme stabilizer (trehalose). The samples tested include free lactate dehydrogenase in buffer supplemented with trehalose (D) and lactate dehydrogenase immobilized in a composition of the invention prepared in buffer without trehalose (C) and in buffer with 10% trehalose (A) or with 5% trehalose (B).

FIG. 5 illustrates the reaction scheme for β-gal reaction with the chromogenic substrate o-nitrophenyl-β-D-galactoside (ONPG).

FIG. 6 illustrates the reaction scheme for β-hydroxybutyrate dehydrogenase (HBDH) catalyzing the oxidation of β-hydroxybutyrate and the reduction of nicotinamide adenine dinucleotide. The subsequent reoxidation of NADH is carried out by an indicator.

FIG. 7 illustrates five different proteins immobilized in a polyurethane hydrogel according to the invention. The proteins are arrayed in a microtiter plate and assayed in parallel. The immobilized proteins are glucose-6-phosphate dehydrogenase (A), alanine dehydrogenase (B), glutamate dehydrogenase (C), lactate dehydrogenase (D), and β-hydroxybutrate dehydrogenase (E). These proteins were assayed against glucose-6-phosphate, alanine, glutamate, lactate, and β-hydroxybutyrate.

FIG. 8 illustrates a diagram for making a protein microarray from an automated procedure that uses a microarrayer.

FIG. 9 illustrates the binding activity of avidin immobilized in a polyurethane-hydrogel composition according to the invention.

FIG. 10 illustrates the binding of a fluorescently-labeled antibody to biotinylated β-galactosidase bound to avidin immobilized in a polyurethane-hydrogel composition according to the invention and treated with biotinylated β-galactosidase.

FIG. 11 illustrates DNA hybridization in a polyurethane-hydrogel composition.

FIG. 12 illustrates an interaction between a protein (transcription factor) immobilized in a polyurethane-hydrogel composition according to the invention and DNA.

FIG. 13 illustrates immobilization of a protein (fibrinogen) after polymerization of a polyurethane-hydrogel composition according to the invention.

FIG. 14 illustrates immobilization of a protein (fibrinogen) after polymerization of a polyurethane-hydrogel composition according to the invention.

FIG. 15 illustrates the reaction scheme catalyzed by cytochrome P450 monooxygenase (CYP1A2) multicomponent enzyme system.

FIG. 16 illustrates the relative nonspecific binding of probes to a polyurethane-hydrogel composition according to the invention. Four polyurethane hydrogels are shown. Two have varying amounts of polyethylenimine and two replace polyethylenimine with other water-soluble crosslinkers suitable for use with the invention. These results illustrate the dependence of nonspecific binding on the amount and selection of the water-soluble crosslinker.

### DETAILED DESCRIPTION OF THE INVENTION

The invention is directed to a method of immobilizing a biologic in a polyurethane-hydrogel composition and to a composition prepared from this method. A composition of the invention includes a biologic immobilized in a network prepared from at least one prepolymer and at least one water-soluble crosslinker. A method of the invention includes admixing a prepolymer, a biologic, and a water-soluble crosslinker in the substantial absence of organic solvent. This admixing step can be stepwise (e.g., derivatizing at least one of the prepolymer and the crosslinker with a biologic and then admixing the derivatized prepolymer with a water-soluble crosslinker or the derivatized crosslinker with a prepolymer or admixing a derivatized prepolymer and a derivatized crosslinker) or concurrent.

An alternative method of the invention includes admixing a prepolymer and a water-soluble crosslinker in the substantial absence of organic solvent and then contacting that mixture with a biologic. This contact can occur anytime during or after polymerization of the prepolymer and the crosslinker. This exposure can be by, for example, admixing the biologic with the reacting prepolymer and the crosslinker, washing a polyurethane hydrogel with a biologic, or spotting a polyurethane hydrogel with a biologic.

A composition of the invention includes an immobilized biologic. According to the invention, the term “immobilized” means that the biologic is fixed to the network formed by admixing a prepolymer and a water-soluble crosslinker and that the biologic is biologically active. Generally the biologic is fixed to the network by covalent interaction, but one skilled in the art will recognize that other interactions (e.g., ionic interactions or entrapment in the network based on size) can also be useful according to the invention. The covalent linkage typically occurs between isocyanate groups of the prepolymer and isocyanate-reactive groups of the biologic such as sulfhydryl (—SH), amino (—NH₂), amido (—CONH₂), hydroxyl (—OH), or carboxyl (—COOH) groups. The reactivity of isocyanate groups with isocyanate-reactive groups is known to one of skill in the art and described in, for example, Polyurethanes: Chemistry and Technology, Volume XVI, Part I, J. H. Saunders and K. C. Frisch eds., pp. 63-128 (1962). One skilled in the art having read this specification will recognize that a biologic can be “immobilized in” or “immobilized on” a composition of the invention. That is, a biologic can be embedded within the network of the polyurethane hydrogel or can be fixed to the surface of the polyurethane hydrogel. These terms can be used interchangeably.

One skilled in the art having read this specification will also recognize that immobilizing agents can also be used to effectuate immobilization of a biologic in a composition of the invention. The term “immobilizing agent” means an electrophilic agent suitable for reacting with an active-hydrogen group available on the water-soluble crosslinker that is used to react with the prepolymer to prepare a polyurethane hydrogel and also suitable for reacting with a biologic. Immobilizing agents are known and readily available. The selection of an immobilizing agent will depend on the biologic that is intended to react with the immobilizing agent and whether the immobilizing agent is intended to react with the prepolymer or the crosslinker. For example, immobilizing agents suitable for reacting with biologics include glutaraldehyde, sulfo-ethylene glycol bis(succinimidylsuccinate), polyoxyethylene bis(glycidylether), dimethyl-3,3′-dithioproprionimidate dihydrochloride, succinic acid maleimidoethyl N-hydroxysuccinimide ester, and 4-(N-maleimidomethyl)cyclohexane-1-carboxylic acid 3-sulfo-N-hydroxysuccinimide ester sodium salt. Immobilizing agents can be particularly useful when a biologic is immobilized in a composition after the composition is polymerized into a polyurethane hydrogel.

By being biologically active, the biologic substantially maintains its properties (e.g., physical and chemical stability and integrity) such that its activity (e.g., hybridization under stringent conditions, respiration, expression, specific-ligating activity, antigenic activity, catalytic activity, oxidative or reductive activity, or binding activity) is comparable to that of the same biologic free in solution or suspension (i.e., not immobilized in a composition prepared by mixing a prepolymer and a water-soluble crosslinker) or in vivo. Various analytical techniques for measuring such activity are known in the art. For example, techniques for measuring protein activity are available in Peptide and Protein Drug Delivery, 247-301, Vincent Lee ed., Marcel Dekker, Inc., New York, N.Y., Pubs. (1991); Enzyme Assays: A Practical Approach, R. Eisenthal and M. Danson eds., Oxford University Press (1993); Methods of Enzymatic Analysis 3^{rd} ed., Bergmeyer et al. eds., Weinheim (1983); and Advanced Drug Delivery Review, 10: 29-90 (1993). Techniques for measuring hybridization of nucleic acids are also available in, for example, Molecular Cloning: Laboratory Manual 3^{rd} ed., J. Sambrook and D. Russell eds., Cold Spring Harbor Laboratory Press (2000). Techniques for measuring cell activity are also known and described in, for example, Industrial Microbiology and Biotechnology 2^{nd} ed., Denain et al. eds. (1999). The particular analytical technique will depend on the selection of the biologic as well as its complementary biospecific agent.

The activity of an immobilized biologic is generally at least great enough to allow for detection of the interaction of the immobilized biologic with its complementary biospecific agent. One skilled in the art will recognize that an effective detectable activity typically depends on the sensitivity of the analytical technique, the selection of the biologic, and the end-use application. For some applications, an activity of about 10% or even about 1% can be an effective detectable activity. The activity of the immobilized biologic is typically at least about 30%, preferably at least about 40%, more preferably at least about 50%, even more preferably at least about 60%, and still more preferably substantially the same as the activity of the corresponding free biologic. The time period for determining this activity will generally depend on the type of biologic and the analytical technique selected.

The term “polymerized” or “polymerizing” means the composition is in the form of gel and does not flow under its own weight. This transformation from liquid components to polymer generally produces molar mass increase, network formation, phase separation, or a combination of these. The flow can be monitored by dispensing the composition (e.g., 200 μL) onto a substrate (e.g., microscope slide) that is positioned perpendicular to gravity and then tipping the substrate such that it is parallel with the force of gravity. The substrate can be coated with a coating compound depending on the substrate selected (e.g., microscope slide coated with polylysine). When the composition does not substantially flow when the substrate is tipped, the composition is considered to be polymerized.

One advantage of the composition of the invention is that it is transparent. According to the invention, the term “transparent” means that a polyurethane-hydrogel composition of the invention is optically transparent such that the polyurethane-hydrogel composition does not substantially interfere with markers such as fluorescent tags or chromatic techniques. This means that a transparent polyurethane-hydrogel composition generally transmits light similar to how water transmits light at the same wavelength. Light transmittance can be determined by the Beer-Lambert Law

$$[ ln ( \frac{{I}_{0}}{I} ) = - ɛ Cl ] ,$$

where ε is the specific molar absorptivity, I is the intensity of transmitted light, Iₒ is the intensity of incident light, l is the film thickness, and C is the concentration of the component with absorptivity ε.

Generally the term “transparent” means that a polyurethane-hydrogel composition of the invention transmits at least about 40 percent, preferably at least about 45 percent, and more preferably at least about 50 percent of light at 600 nanometers (nm) through a quartz cuvette having a cell pathlength of about 4 cm. One skilled in the art knows that transmittance varies with wavelength and pathlength and that 600 nm represents the middle of the visible spectrum, which ranges between about 400 nm and 800 nm.

In one embodiment, a transparent polyurethane-hydrogel composition of the invention transmits at least about 1.5 times, preferably at least about 2 times, more preferably at least about 3 times, and still more preferably at least about 4 times more light at 600 nm through a quartz cuvette having a cell pathlength of about 4 cm than a gel composition prepared from a crosslinker (e.g., water, ethylene diamine, diethylene diamine, and triethylene triamine) other than a water-soluble crosslinker according to the invention. Such a comparison uses the same testing conditions—e.g., time, wavelength, cell thickness, and temperature—for each sample.

The percent transmission can be determined within at least about 36 hours of preparing a composition, preferably within at least about 24 hours of preparing a composition, and more preferably within at least about 12 hours of preparing a composition. Deionized water at about pH 7 can be used as the control.

A polyurethane hydrogel of the invention has physical gel properties suitable for its intended end-use application. These physical properties can be modified by selection of the amount and type of hydrogel components, particularly isocyanate, polyol, and water-soluble crosslinker.

One such property is crosslink density. Crosslink density affects the stiffness, tensile modulus, and compressive strength of a material. One of skill in the art is familiar with these relationships, but they will be briefly described here.

$$Crosslink denisty = \frac{number of crosslinks}{polymer mass} \qquad ( 1 )$$

The molecular weight between crosslinks of a system, M_{c}, will also be related to the crosslink density of a system. M_{c} is related to the density of the material by the approximation shown in Equation (2). This parameter is related to the Shear Modulus of the system. via Equation (3) and to the Young's Tensile Modulus by Equation (4).

$$ρ \approx \frac{{NM}_{c}}{{N}_{A}} \qquad ( 2 )$$

where:
N is the number of chains per unit volume
N_{A} is Avogadro's number

$$G = ρ \frac{RT}{{M}_{c}} \qquad ( 3 )$$

where:
G is the shear modulus
ρ is the density of the dry network (≈1 gm/cc)
R is the gas constant
T is the temperature and
M_{c} is the average molecular weight between elastically effective crosslinks
The Young's Tensile Modulus, E, is given by:

$$E = \frac{3 ρ RT}{{M}_{c}} = 3 G \qquad ( 4 )$$

One physical property is based on the number-average molecular weight between crosslinks (M_{c}). An effective M_{c} provides support to a three-dimensional gel configuration and provides a substantially stable gel, and the M_{c} generally is not so great or so low that a composition of the invention becomes unsuitable for its intended end-use application. A composition of the invention is unsuitable for its intended end-use application if, for example, a biospecific agent cannot diffuse into the network of a polyurethane hydrogel to interact with an immobilized biologic.

The number-average molecular weight between crosslinks can be measured experimentally by swelling the gel and measuring the gel's change in volume-mass ratio. The value of number-average molecular weight between crosslinks can be controlled by varying the amount and molecular weight of prepolymer and the amount and molecular weight of water-soluble crosslinker. The nature of the gel and its internal topology can be varied, and even optimized, by simulation of gelation through the use of Monte Carlo gelation-simulation techniques. These techniques allow for an estimate of gel characteristics including such measures as the crosslink density of the network as well as the number-average molecular weight between crosslinks.

According to simulation techniques, a composition according to the claimed invention generally has an M_{c} of at least about 2,000, preferably at least about 3,000, more preferably at least about 4,000, and still more preferably at least about 5,000. But the M_{c} is generally no greater than about 8,000, preferably no greater than about 7,000, and more preferably no greater than about 6,000.

These M_{c} values may be related to experimental observables—e.g., the tensile modulus. An effective tensile modulus for a composition of the invention is great enough to provide a shape suitable for an end-use application. For a composition of the invention, the tensile modulus can be difficult to measure due to its low value, but the tensile modulus can be reliably estimated from the number-average molecular weight between crosslinks.

Generally a transparent polyurethane hydrogel of the invention has a tensile modulus of at least about 800 kiloPascal (kPa), preferably at least about 1200 kPa, and more preferably at least about 1500 kPa at a temperature of about 25° C. Generally the tensile modulus is no greater than about 4000 kPa, preferably no greater than about 3000 kPa, and more preferably no greater than about 2000 kPa at a temperature of about 25° C.

The terms “desirable physical properties” and “desirable physical property” mean desirable values for number-average molecular weight between crosslinks or tensile modulus as described above.

Also according to the invention, the term “polyurethane” can refer to polyurethane, polyurea, or a mixture of polyurea and polyurethane. A polyurethane material can be obtained by a reaction of a polyol with an isocyanate. A polyurea material can be obtained by reaction of an amine with an isocyanate. A polyurethane material or a polyurea material can contain both urea functionality and urethane functionality, depending on the components included in a composition. Preferably a composition of the invention is a mixture of polyurethane material and polyurea material, which is generally known as a polyureaurethane. For purposes of this specification, no further distinction will be made between polyurethane and polyurea.

A composition of the invention is prepared in an aqueous solution and in the substantial absence of an organic solvent. The terms “substantially free of organic solvent” and “substantial absence of organic solvent” mean an amount of organic solvent insufficient for dispersing hydrogel components to induce transparency in a polyurethane-hydrogel composition of the invention. This amount can include trace amounts of organic solvent but not so much organic solvent that a biologic would be sufficiently denatured or inactivated as to prevent or substantially retard a detectable interaction with a biospecific agent.

Generally the amount of organic solvent is no more than about 3 weight percent, preferably no more than about 2 weight percent, more preferably no more than about 1 weight percent, and even more preferably no more than about 0.5 weight percent. Still more preferably, the amount of organic solvent is no more than about 0.1 weight percent. Examples of organic solvents include acetonitrile, dimethyl formamide, dimethyl sulfoxide, tetrahydrofuran, dioxane, dichloromethane, acetone, and methyl ethyl ketone. The term “weight percent” is based on the total weight of the hydrogel components that are used to prepare a transparent polyurethane-hydrogel composition of the invention. The balance of all formulations is aqueous solvent.

The term “hydrogel component(s)” includes any component used to prepare a polyurethane-hydrogel composition of the invention such as isocyanate, polyol, aqueous solvent, biologic, water-soluble crosslinker, and biologic-stabilizer additives, for example, antioxidant, antifreeze, preservative, chelator, lyoprotectant, and any other additive suitable for stabilizing or maintaining the activity of a cell or protein.

The term “composition” or “polyurethane-hydrogel composition” will be understood to one of skill in the art having read this specification. To form a gel-based formulation, hydrogel components are mixed together. Initially much of the components will be dispersed in solution, but as the components begin to react to completion (i.e., polymerize), a gel network having solvent molecules dispersed throughout the network will form. Thus, a “composition” of the invention includes a polymerized composition (i.e., the reaction product of hydrogel components when the gel network is formed), but the “composition” also includes a reaction mixture when the hydrogel components and biologic are initially introduced and before a network is substantially formed. The term “polyurethane hydrogel” can be used to specifically refer to a composition that is polymerized.

A composition of the invention is particularly useful for biomedical applications, such as assays useful for diagnostic devices and therapeutic applications. One particularly useful application is a protein microarray or a protein array. Another particularly useful application is a cell microarray or a cell array.

Polyurethane-Hydrogel Composition

A composition of the invention is generally prepared by admixing at least one isocyanate, at least one polyol, at least one water-soluble crosslinker, and at least one biologic in aqueous solution. A composition of the invention can alternatively be prepared by admixing a prepolymer and a water-soluble crosslinker in an aqueous solvent and in the substantial absence of organic solvent and then contacting that mixture with a biologic. This contact can occur anytime during or after polymerization of the prepolymer and the crosslinker. This contact can be by, for example, admixing the biologic with the reacting prepolymer and the crosslinker, washing a polyurethane hydrogel with a biologic, or spotting a polyurethane hydrogel with a biologic.

As another example, the contact can be provided by derivatizing at least one of a crosslinker and a prepolymer with a biologic and then admixing the derivatized crosslinker, derivatized prepolymer, or a combination of these with other hydrogel components. The terms “derivatize,” “derivatizing,” and “derivatized” mean any method suitable for fixing the biologic to a hydrogel component or polyurethane hydrogel. Such methods include creating covalent linkages between an isocyanate group of the prepolymer and the isocyanate-reactive groups of the biologic and creating covalent linkages between an immobilizing agent, a biologic, and at least one of a prepolymer and a water-soluble crosslinker.

Preferably the isocyanate and the polyol are introduced in the form of a prepolymer.

These components are chosen such that upon preparing a polyurethane hydrogel of the invention, the polyurethane hydrogel has desirable physical properties for the intended application, the polyurethane hydrogel is transparent, and the biologic can be immobilized in the polyurethane hydrogel.

A composition of the invention can also include additives that are known to be useful in polyurethane-hydrogel compositions for intended end-use applications or are known to promote stability of a biologic.

Prepolymer

A polyurethane-hydrogel composition of the invention includes a prepolymer. Any prepolymer suitable for preparing a transparent polyurethane-hydrogel composition and an immobilized biologic can be used.

The prepolymer is generally present in an amount effective for providing a transparent polyurethane hydrogel with a desirable physical property. This amount should not be so great that the polyurethane hydrogel is not transparent and not so low that the polyurethane hydrogel does not have a desirable physical property.

The prepolymer is generally present in an amount of no more than about 5 weight percent, preferably no more than about 4.5 weight percent, and preferably no more than about 4 weight percent. But the prepolymer is generally present in an amount of at least about 1 weight percent, preferably at least about 1.5 weight percent, and more preferably at least about 2 weight percent. In one embodiment, the prepolymer is present in an amount of between about 2.5 weight percent and about 3.5 weight percent. In another embodiment, the prepolymer is present in an amount of about 3 weight percent.

A prepolymer suitable for use with the invention generally includes a reaction product of at least one water-soluble polyol and at least one isocyanate. One skilled in the art having read this specification would understand that isocyanates and polyols that promote water solubility of the prepolymer and that do not substantially adversely affect transparency of the composition or immobilization of a biologic would be desirable.

The term “polyol” refers to a compound that has two or more isocyanate-reactive functional groups per molecule. These functional groups include hydroxyl (—OH), sulfhydryl (—SH), carboxyl (—COOH), and amino (—NHR, with R being hydrogen, an alkyl moiety of C₁ to C₁₀, or epoxy) groups. The functional group is preferably —OH. The term “polyol” includes diol.

A water-soluble polyol suitable for use in the invention includes polyoxyalkylene polyols or polyols made up of ethylene-oxide monomer units. For polyols made up of ethylene-oxide monomer units, at least 75 weight percent, preferably at least 90 weight percent, and more preferably at least 95 weight percent of the units should be ethylene oxide. Even 100 weight-percent ethylene oxide-containing polyols can be used. These polyols can include up to about 25 weight-percent propylene-oxide monomer units.

The water-soluble polyol generally has an average molecular weight of at least about 2,000, preferably at least about 5,000, and more preferably at least about 7,000 gram/mole. But the molecular weight generally is no greater than about 30,000, preferably no greater than about 20,000, more preferably no greater than about 15,000, and still more preferably no greater than about 10,000 gram/mole. In one embodiment, the water-soluble polyol has a molecular weight of about 7,500 gram/mole.

Suitable polyols include diols such as a high molecular-weight polyethyleneglycol of the formula H(OCH₂CH₂)ₓOH where x is an average number such that the glycol has an average molecular weight of at least about 500, preferably at least about 1,000, and more preferably at least about 2,000 gram/mole. But the average molecular weight of the glycol generally is no greater than about 30,000, preferably no greater than about 20,000, more preferably no greater than about 15,000, and still more preferably no greater than about 10,000 gram/mole.

Preferably the polyol includes at least one triol (i.e., trihydroxy compound) and is synthesized using initiators such as glycerol, trimethylolpropane, and triethanolamine.

Other polyols having more than 3 functional groups are also suitable and can be synthesized using initiators such as sorbitol, erythritol, sucrose, and pentaerythritol. These initiators can be used to make polyoxyalkylene polyols as well as polyols made up of ethylene-oxide monomer units.

Suitable polyoxyalkylene polyols include polyols that have at least one oxyethylene, oxypropylene, or oxybutylene repeat unit. Examples include polyoxypropylene glycols (e.g., VORANOL P-2000 polyol and VORANOL P-4000 polyol, both trademarks of, and available from, The Dow Chemical Company); polyoxypropylene-oxyethylene glycols (e.g., DOWFAX DM-30 surfactant and SYNALOX 25 D-700 polyglycol, both trademarks of, and available from, The Dow Chemical Company); polyoxyethylene triols (e.g., TERRALOX WG-98 lubricant and TERRALOX WG-116 lubricant, both trademarks of, and available from, The Dow Chemical Company); polyoxypropylene-oxyethylene triols (e.g., VORANOL CP 1000 polyol, VORANOL CP 3055 polyol, VORANOL CP 3001 polyol, and VORANOL CP 6001 polyol, all trademarks of, and available from, The Dow Chemical Company); and polyoxyethylene hexols (e.g., TERRALOX HP-400 lubricant, trademark of, and available from, The Dow Chemical Company).

Suitable polyols made up of ethylene-oxide monomer units include polyols made from initiators reacted with ethylene oxide.

Functionality of the polyol is effective to facilitate processability of a prepolymer of the invention. The functionality should not be so low that a composition of the invention can take an undesirable amount of time to gel. But the functionality should not be so high that it substantially adversely effects gel time, transparency, or physical properties of the polyurethane hydrogel.

According to the invention, a polyol can have a functionality of at least about 2, preferably at least about 3, more preferably at least about 4, and even more preferably at least about 5. Generally the functionality is no greater than about 10, preferably no greater than about 9, and more preferably no greater than about 8.

In one embodiment, the functionality is at least 3. In another embodiment, the functionality is between about 2 and about 5.

Preferably the polyol is a 7,000 molecular-weight triol copolymer of ethylene oxide (75%) and propylene oxide (25%) (e.g., PLURACOL VY polyol and PLURACOL 1123 polyol trademark of, and available from, BASF, Mount Olive, N.J.).

A prepolymer according to the invention includes an isocyanate. Any isocyanate suitable for preparing a transparent polyurethane-hydrogel composition having an immobilized biomolecule can be used. One skilled in the art having read the specification would understand that the selection of the isocyanate will depend on such factors as the selection of the polyol, the degree of handling or shaping used to prepare the polyurethane-hydrogel composition, and the end-use application of the composition.

The isocyanate can be advantageously selected from at least one of an organic isocyanate or at least one of a multifunctional polyisocyanate. These include aliphatic isocyanates and cycloaliphatic isocyanates. Examples of aliphatic isocyanates and cycloaliphatic isocyanates include hexamethylene diisocyanate; trans, trans-1,4-cyclohexyl diisocyanate; 2,4-and 2,6-hexahydrotoluenediisocyanate; 4,4′-,2,2′-,2,4′-dicyclohexylmethanediisocyanate; 1,3,5-tricyanato cyclohexane; isophorone diisocyanate trimers; and isophorone diisocyanate. Preferably the isocyanate is isophorone diisocyanate.

Although less preferred because they can discolor over time, the isocyanate can also include aromatic isocyanates. Examples of aromatic isocyanates include toluene-2,4-diisocyanate; toluene-2,6-diisocyanate; commercial mixtures of toluene-2,4 and 2,6-diisocyanates; m-phenylene diisocyanate; 3,3′-diphenyl-4,4′-biphenylene diisocyanate; 4,4′-biphenylene diisocyanate; 4,4′-diphenylmethane diisocyanate; 3,3′-dichloro-4,4′-biphenylene diisocyanate; cumene-2,4-diisocyanate; 1,5-napthalene diisocyanate; p-phenylene diisocyanate; 4-methoxy-1,3-phenylene diisocyanate; 4-chloro-1,3-phenylene diisocyanate; 4-bromo-1,3-phenylene diisocyanate; 4-ethoxy-1,3-phenylene diisocyanate; 2,4-dimethyl-1,3-phenylene diisocyanate; 5,6-dimethyl-1,3-phenylene diisocyanate; 2,4-diisocyanatodiphenylether; 4,4′-diisocyanatodiphenylether benzidine diisocyanate; 4,6-dimethyl-1,3-phenylene diisocyanate; 9,10-anthracene diisocyanate; 4,4′-diisocyanatodibenzyl; 3,3′-dimethyl-4,4′-diisocyanatodiphenylmethane; 2,6-dimethyl-4,4′-diisocyanatodiphenyl; 2,4-diisocyanatostilbene; 3,3′-dimethoxy-4,4′-diisocyanatodiphenyl; 1,4-anthracenediisocyanate; 2,5-fluorenediisocyanate; 1,8-naphthalene diisocyanate; 2,6-diisocyanatobenzfuran; 2,4,6-toluene triisocyanate; p,p′p″-triphenylmethane triisocyanate; and polymeric 4,4′-diphenylmethane diisocyanate.

A composition of the invention generally includes isocyanate in an amount effective for providing a desirable tensile modulus or a desirable number-average molecular weight between crosslinks. This amount should not be so high that a prepolymer becomes unprocessable or so low that the tensile modulus or number-average molecular weight between crosslinks of a polyurethane hydrogel is substantially adversely affected.

A prepolymer according to the invention generally includes an isocyanate-to-hydroxyl (NCO/OH) site or group (i.e., moles NCO×functionality/moles crosslinker×functionality) ratio of at least about 2, preferably at least about 2. 1, and more preferably at least about 2.2. But this ratio is generally no greater than about 4, preferably no greater than about 3, and more preferably no greater than about 2.5.

A prepolymer according to the invention is generally dispersed in aqueous solvent to form an aqueous prepolymer solution. An aqueous prepolymer solution desirably has a viscosity effective for processing a composition according to the invention. This solution can also contain additives that facilitate solubility of the prepolymer so long as the additives are not substantially incompatible with the components in a composition of the invention.

A prepolymer according to the invention can be prepared by methods known in the art and can be obtained commercially. Known methods for preparing a prepolymer according to the invention generally involve admixing a polyol with an isocyanate and heating the mixture to a temperature effective to facilitate the reaction between the polyol and isocyanate. Examples of prepolymers suitable for use according to the invention, as well as methods for making such prepolymers, are included in U.S. Pat. No. 5,462,536. One such prepolymer is Hypol G-50 hydrophilic polymer (a trademark of The Dow Chemical Company, Midland, Mich.), which is describe in Example 1 of this specification. One of skill in the art will also appreciate that the age of a prepolymer (i.e., the amount of time that passes between initial formation of the prepolymer and when the prepolymer is incorporated into a polyurethane-hydrogel composition) may affect the molecular weight of the prepolymer, which in turn, may affect how a particular prepolymer affects a polyurethane-hydrogel composition of the invention. One of skill in the art will also readily recognize that it may be less preferred to use a prepolymer immediately after it is prepared (i.e., fresh prepolymer), and it may be preferred to allow the prepolymer to build some additional molecular weight before incorporating it into a composition of the invention. This phenomenon is known in the polymer field, and one of skill in the art can readily determine the optimal age of a prepolymer without undue experimentation.

Biologic

A biologic can be immobilized in a composition of the invention. The prepolymer, the water-soluble crosslinker, or a combination of these can be derivatized by the biologic to immobilize the biologic in a composition of the invention. Alternatively, a polyurethane hydrogel according to the invention can be contacted with a biologic to immobilize the biologic in the polyurethane hydrogel.

The term “biologic” generally includes biopolymers and cells.

The term “biopolymer” includes peptides, nucleics, and peptide nucleic acids. The term “biopolymer” also includes saccharides (e.g., oligo- and polysaccharides); lipopolysaccharides; glycolipids; and combinations or hybrids of these. A biopolymer further includes combinations and hybrids of any biopolymer with any other biopolymer. A biopolymer can be synthetic, native to a living organism (e.g., human; animal; plant; protis; fungus such as yeast; bacterium including mycoplasm and nanobe; or archaeon), native to a virus or bacteriophage, or genetically engineered. Native biopolymers include functional derivatives of biopolymers. A biopolymer is preferably water soluble—i.e., dispersible in aqueous solution that is substantially free of organic solvent. But a biopolymer can also be dispersible in aqueous solvent by use of a dispersing aid such as a surfactant.

One skilled in the art will recognize that biopolymers suitable for use according to the invention can be formulated from techniques that are known in the art, including synthetic techniques (e.g., recombinant techniques and peptide synthesis) or can be isolated from an endogenous source of the biopolymer. A native sequence refers to a sequence that occurs in nature in any cell type whether purified from a native source, synthesized, produced by recombinant DNA technology, or by any combination of these methods. A functional derivative refers to a sequence that has a qualitative biological activity in common with the native sequence.

The term “nucleics” include oligonucleotides and polynucleotides. Nucleics include single- or multiple-stranded configurations. For multiple-stranded configurations, one or more of the strands may or may not be completely aligned with another.

An “oligonucleotide” generally refers to a nucleotide multimer of about 10 to 100 nucleotides in length, while a “polynucleotide” includes a nucleotide multimer having any number of nucleotides. A nucleotide refers to a subunit of a nucleic acid and includes a phosphate group, a 5-carbon sugar and a nitrogen-containing base as well as analogs of such subunits. A polynucleotide particularly includes DNA (including cDNA), RNA, binding polynucleotides (e.g., optomers), and catalytic polynucleotides (e.g., RNAzymes). A polynucleotide includes those compounds in which the conventional polynucleotide backbone has been replaced with a non-naturally occurring or synthetic backbone and also includes nucleic acids in which one or more of the conventional bases has been replaced with a synthetic base capable of participating in Watson-Crick type hydrogen-bonding interactions.

“Peptide nucleic acids” include analogues of DNA in which the backbone is a pseudopeptide rather than a sugar. PNA can mimic DNA behavior and bind complementary polynucleotide or oligonucleotide strands. PNAs are described in, for example, Peptide Nucleic Acids. Protocols and Applications, P. E. Nielsen and M. Egholn eds. (1999).

“Peptides” include compounds made from alpha amino acids being joined together through amide bonds. Peptides include dipeptides, tripeptides, oligopeptides, and polypeptides. Polypeptides are polymers of amino acids and include proteins. In the context of this specification, it should be appreciated that the amino acids can be the L-optical isomer or the D-optical isomer and include synthetic amino acids.

Proteins generally include any sequence of amino acids for which the primary and secondary structure of the sequence is sufficient to produce higher levels of tertiary and/or quaternary structure. Proteins are distinct from peptides that do not have such structure. Proteins typically have a molecular weight of at least about 15 kilodaltons.

Examples of proteins include nucleic-acid regulatory and storage proteins (e.g., DNA-binding proteins, transcription factors, zinc-finger proteins, repressors, and histones); immunoproteins and other recognition and/or signaling proteins (e.g., antibodies, catalytic antibodies such as abzymes, lectins, hormones, cytokines, and growth factors); integral membrane proteins (e.g., photosynthetic-reaction center and electron-transfer proteins, cell pore proteins, cell-surface glycoproteins, proton and ion pump proteins, and voltage-gated channel and junction proteins); structural proteins (e.g., actin, myosin, collagen, fibrin, keratin, silk proteins, proteoglycans and adhesion proteins, cell-wall glycoproteins, and viral envelope and capsid proteins); specialized-binding, storage, and/or transport proteins (e.g., lipoproteins, ferritin, albumins, avidin, hemoglobins, myoglobins, translation factors, export system proteins, and various periplasmic and mitochondrial matrix proteins); chaperonins; disease-causing and disease-inhibiting proteins (e.g., prions, protein toxins, and peptide antibiotics); and enzymes.

Enzymes include oxidoreductases (EC 1: e.g., monooxygenases, cytochromes, dioxygenases, dehydrogenases, metalloreductases, ferredoxin, and thioredoxin); transferases (EC 2: e.g., glycosyltransferases, alkyltransferases, acyltransferases, carboxyltransferases, fatty acyl synthases, kinases, RNA and DNA polymerases, and reverse transcriptases); hydrolases (EC 3: e.g., glycosylases, glycosidases, peptidases and proteases, nucleases, phosphatases, and lipases); lyases (EC 4: e.g., decarboxylases, RUBISCO, and adenylate cyclase); isomerases (EC 5: e.g., racemases, epimerases, mutases, topo-isomerases, and foldases); and ligases (EC 6: e.g., carboxylases and acyl synthetases).

In certain embodiments, the protein is an antibody. The antibody may bind to, for example, any of the above-mentioned molecules. Exemplary molecular targets for antibodies encompassed by the invention include CD proteins such as CD3, CD4, CD8, CD19, CD20, and CD34; members of the HER receptor family such as the EGF receptor, HER2, HER3, or HER4 receptor; cell-adhesion molecules such as LFA-1, Mol, p150, VLA-4, ICAM-1, VCAM, and av/p3 integrin including either α or β subunits (e.g., anti-CD11a, anti-CD18, or anti-CD11b antibodies); growth factors such as VEGF; IgE; blood group antigens; flk2/flt3 receptor; obesity (OB) receptor; and protein C.

The term “antibody” is used in the broadest sense and specifically covers native and genetically-engineered monoclonal and polyclonal antibodies (including full-length antibodies that have an immunoglobulin Fc region), antibody compositions with polyepitopic specificity, bispecific antibodies, diabodies, triabodies, and single-chain molecules as well as antibody fragments (e.g., Fab, F(ab′)₂, and Fv).

The term “monoclonal antibody” refers to an antibody obtained from a population of substantially homogeneous antibodies (i.e., the individual antibodies in the population are identical except for possible naturally-occurring mutations that may be present in minor amounts). Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by the hybridoma method first described by Kohler et al., Nature, 256: 495 (1975) or may be made by recombinant DNA methods (see, e.g., U.S. Pat. No. 4,816,567). The monoclonal antibodies may also be isolated from phage antibody libraries using the techniques described in, for example, Clackson et al., Nature, 352:624-628 (1991) and Marks et al., J. Mol. Biol., 222:581-597 (1991).

Monoclonal antibodies specifically include chimeric antibodies (immunoglobulins) in which a portion of the heavy and/or light chain is identical to or homologous to corresponding sequences in antibodies derived from a particular species or belonging to a particular antibody class or subclass, while the remainder of the chain(s) is identical to or homologous to corresponding sequences in antibodies derived from another species or belonging to another antibody class or subclass as well as fragments of such antibodies, so long as they exhibit the desired biological activity (see e.g., U.S. Pat. No. 4,816,567; Morrison et al., Proc. Natl. Acad. Sci. USA, 81:6851-6855 (1984)).

Monoclonal antibodies also include humanized forms of nonhuman (e.g., murine) antibodies, which are chimeric immunoglobulins or immunoglobulin chains or fragments (such as Fv, Fab, Fab′, F(ab′)₂, or other antigen-binding subsequences of antibodies) that contain minimal sequence derived from nonhuman immunoglobulin. For the most part, humanized antibodies are human immunoglobulins (recipient antibody) in which residues from a complementarily-determining region (CDR) of the recipient are replaced by residues from a CDR of a nonhuman species (donor antibody) such as mouse, rat, or rabbit having the desired specificity, affinity, and capacity. In some instances, Fv framework-region (FR) residues of the human immunoglobulin are replaced by corresponding nonhuman residues. Furthermore, humanized antibodies can comprise residues that are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a nonhuman immunoglobulin and all or substantially all of the FR regions are those of a human immunoglobulin sequence. The humanized antibody optimally also will include at least a portion of an immunoglobulin constant region (Fc), typically that of a human immunoglobulin. For further details, see Jones et al., Nature, 321:522-525 (1986); Reichmann et al., Nature, 332:323-329 (1988); and Presta, Curr. Op. Struct. Biol., 2:593-596 (1992). The humanized antibody includes a PRIMATIZED antibody (trademark of, and available from, IDEC Pharmaceutical Corp., San Diego, Calif.) in which the antigen-binding region of the antibody is derived from an antibody produced by immunizing macaque monkeys with the antigen of interest.

One skilled in the art having read this specification will recognize that proteins can include, but need not include, proteins that rely on cofactors to effectuate activity or proteins that can interact with other proteins in a system such as in a bienzyme system or in a multienzyme pathway. Such systems can also include cofactors to effectuate activity.

For example, lactate dehydrogenase can rely on NAD⁺ (nicotinamide adenine dinucleotide) to effectuate activities. Cofactors can be added to a composition of the invention or can be provided by contact with a polyurethane hydrogel by, for example, washing the polyurethane hydrogel with an aqueous solution containing the cofactor.

As another example, a multienzyme system can include at least two enzymes and can also include organic cofactors, inorganic cofactors, cosubstrates, or other reactants specific to the multienzyme system employed. Generally such a system allows a first enzyme to react with an enzyme substrate to form a product, and the second enzyme can react with that product either to regenerate the initial enzyme substrate or to form a further derivative of that product. One example of a bienzyme system includes lactate dehydrogenase and diaphorase, and the cosubstrates NAD⁺ and DCIP (2,6-dichloroindolphenol).

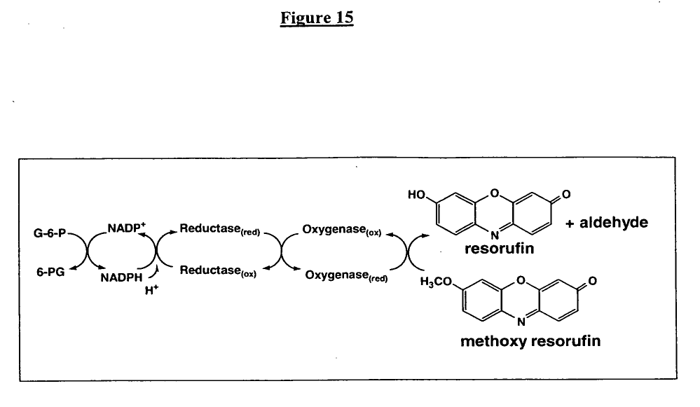

Bienzyme systems or multienzyme systems can be added to a composition of the invention or can be provided by immobilizing at least one component of the system in a polyurethane hydrogel and then contacting the polyurethane hydrogel with the remaining components by, for example, washing the polyurethane hydrogel with an aqueous solution containing the remaining components. One example of a multienzyme system is a reaction scheme that uses cytochrome P450 monooxygenase as shown in FIG. 15.

The term “cells” includes a variety of eukaryotic and prokaryotic cells and includes human, animal (e.g., mammalian), plant, protist, fungal (e.g., yeast), bacterial (including mycoplasms and nanobes), archaea, protoplasts, cytoplasts, membrane-bound cell fragments, and liposomes. Cells can be native or genetically engineered.

Although this invention is not limited to any particular theory, it is believed that the cells are immobilized in a composition of the invention by reaction of an isocyanate group and an isocyanate-reactive group available on the surface of the cell such as a protein or a polysaccharide available on the surface of the cell.

Examples of suitable bacterial cells include Gram-positive (e.g., genera of Bacillus, Mycobacterium, and Rhodococcus) and Gram-negative (e.g., genera of Escherichia, Pseudomonas, and Agrobacterium) bacterial cells.

Examples of suitable fungal cells include genera of Saccharomyces and Asperigillus.

One type of cell that is particularly useful according to the invention includes host cells. Host cells include any cell suitable for being transformed with an expression vector constructed using recombinant DNA techniques. An expression vector includes any vector that is capable of expressing a DNA sequence contained in the vector when the DNA sequence is operably linked to other sequences capable of effecting its expression. Expression vectors are generally found in the form of a plasmid, which is a circular double-stranded DNA that is not bound to a chromosome. The terms “plasmid” and “vector” can be used interchangeably. One skilled in the art generally understands host cells including use of host cells, construction of host cells, and expression of host cells. A brief description will be provided here for illustration only and is not meant to be limiting.

The vectors and methods disclosed here are suitable for use in host cells over a range of prokaryotic and eukaryotic organisms.

Prokaryotes can be used for expression. In general, plasmid vectors containing replicon and control sequences that are derived from a species compatible with the host cell are used in connection with these hosts. The vector ordinarily carries a replication site as well as marking sequences that are capable of providing phenotypic selection in transformed cells. For example, E. coli is typically transformed using pBR322, which is a plasmid derived from an E. coli species (see, e.g., Bolivar et al., Gene, 2: 95 (1977)). PBR322 contains genes for ampicillin and tetracycline resistance and thus provides easy means for identifying transformed cells. The pBR322 plasmid or microbial plasmid must also contain, or be modified to contain, promoters that can be used by the microbial organism for expression of its own proteins. Those promoters most commonly used in recombinant DNA construction include the β-lactamase (penicillinase) and lactose promoter systems (see, e.g., Chang et al., Nature, 275: 617 (1978); Itakura et al., Science, 198: 1056,(1977); (Goeddel et al., Nature, 281: 544 (1979)) and a tryptophan (trp) promoter system (see, e.g., Goeddel et al., Nucleic Acids Res., 8: 4057 (1980); and EPO Application Publication No. 0036776). While these are the most commonly used, other microbial promoters have been discovered and utilized, and details concerning their nucleotide sequences have been published, enabling a skilled worker to ligate them functionally with plasmid vectors (see, e.g., Siebenlist et al., Cell, 20: 269 (1980)).

In addition to prokaryotes, eukaryotic microbes, such as yeast cultures can also be used. Saccharomyces cerevisiae, or common baker's yeast, is the most commonly used among eukaryotic microorganisms, although a number of other strains are commonly available. For expression in Saccharomyces, the plasmid YRp7, for example, (see, e.g., Stinchcomb et al., Nature, 282: 39 (1979); Kingsman et al., Gene, 7: 141 (1979); and Tschemper et al., Gene, 10; 157 (1980)) is commonly used. This plasmid already contains the trp 1 gene which provides a selection marker for a mutant strain of yeast lacking the ability to grow in tryptophan, for example ATCC No. 44076 or PEP4-1 (see, e.g., Jones, Genetics, 85: 12 (1977)). The presence of the trp1 lesion as a characteristic of the yeast host cell genome then provides an effective environment for detecting transformation by growth in the absence of tryptophan.

Suitable promoting sequences in yeast vectors include the promoters for 3-phosphoglycerate kinase (see, e.g., Hitzeman et al., J. Biol. Chem., 255: 12073 (1980)) or other glycolytic enzymes (see, e.g., Hess et al., J. Adv. Enzyme Reg., 7: 149 (1968); and Holland et al., Biochemistry, 17: 4900 (1978)), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the mRNA and termination. Other promoters, which have the additional advantage of transcription controlled by growth conditions, are the promoter regions for alcohol dehydrogenase 2, isocytochrome C, acid phosphatase, degradative enzymes associated with nitrogen metabolism, glyceraldehyde-3-phosphate dehydrogenase, and enzymes responsible for maltose and galactose utilization. Any plasmid vector containing a yeast-compatible promoter, origin of replication, and termination sequences is suitable.

Cultures of cells derived from multicellular organisms can also be used as hosts. These cell cultures can be from a vertebrate or an invertebrate culture. But interest has been greatest in vertebrate cells, and propagation of vertebrate cells in culture (tissue culture) has become a routine procedure in recent years (see, e.g., Tissue Culture, Academic Press, Kruse and Patterson eds. (1973)). Examples of such useful host cell lines are VERO and HeLa cells, Chinese hamster-ovary (CHO) cell lines, and W138, BHK, COS-7, and MDCK cell lines. Expression vectors for such cells ordinarily include (if necessary) an origin of replication, a promoter located in front of the gene to be expressed, along with any necessary ribosome-binding sites, RNA splice sites, polyadenylation site, and transcriptional terminator sequences.

For use in mammalian cells, the control functions on the expression vectors are often provided by viral material. For example, commonly used promoters are derived from polyoma, Adenovirus 2, and most frequently Simian Virus 40 (SV40). The early. and late promoters of SV40 virus are particularly useful because both are obtained easily from the virus as a fragment which also contains the SV40 viral origin of replication (see, e.g., Fiers et al, Nature, 273: 113 (1978). Smaller or larger SV40 fragments may also be used so long as there is a 250 bp sequence extending from the Hind III site toward the Bgl I site located in the viral origin of replication. It is also possible, and often desirable, to utilize promoter or control sequences normally associated with the desired gene sequence so long as control sequences are compatible with the host-cell systems.

An origin of replication can be provided either by construction of the vector to include an exogenous origin, such as can be derived from SV40 or other viral (e.g. Polyoma, Adeno, VSV, BPV, etc.) source, or can be provided by the host cell chromosomal replication mechanism. If the vector is integrated into the host-cell chromosome, the latter is often sufficient.

Transfection can be carried out by, for example, calcium-phosphate precipitation (see, e.g., Graham and Van der Eb, Virology, 52: 456 (1973)), nuclear injection, protoplast fusion, and calcium treatment using calcium chloride (see, e.g., Cohen et al., Proc. Natl. Acad. Sci. (USA), 69: 2110 (1972)).

Suitable vectors containing the desired coding and control sequences can be made by standard ligation techniques, and isolated plasmids or DNA fragments can be cleaved, tailored, and religated to form desirable plasmids.

The biologic is generally selected to effectuate an intended end-use application. That is, the biologic is generally selected for an anticipated selective interaction between a biospecific agent and the biologic. A biospecific agent includes any molecule (e.g., small molecules, proteins, nucleics, and ligands) that can be complementary to a biologic. Pairs for selective interactions include protein-protein, antigen-antibody, substrate-enzyme, effector-enzyme, inhibitor-enzyme, complementary nucleic-acid strands, ligand-binding molecule, and plasmid-host cell. Thus, the biologic is selected based on the interaction to be analyzed. For example, if the biologic is an antigen, an antibody is expected as the biospecific agent.

The selective interactions can be used to analyze, for example, binding affinities, catalysis of reactions, proteolysis of substrates, inhibition of enzymes, expression of proteins, metabolic pathways, and other biological processes.

The biologic is added in an amount effective to provide detectable interaction with its complementary biospecific agent when exposed to a sample containing the biospecific agent. This amount should not be so great that the biologic interferes with gel formation or activity analysis or so low that the interaction between the biologic and its complementary biospecific agent is not detectable. Generally an effective amount of biologic will depend on the type of biopolymer selected and the end-use application. This amount can also depend on the sensitivity of the activity technique selected for analysis. One of skill in the art having read this specification would understand how to select an effective amount of biologic.

A desirable amount of biologic can depend on the number of isocyanate-reactive groups (i.e., functionality) available to react with the isocyanate. The functionality of, for example, a protein can depend on the specific protein, the conformation of the protein, and the reaction conditions such as pH and ionic strength.

It will be appreciated that a biologic can also be covalently modified by known methods. This modification can be introduced before or after immobilizing the biopolymer in a composition of the invention. Any modification that is not substantially incompatible with the composition of the invention is suitable. It can be desirable to modify a biologic, for example, to introduce isocyanate-reactive groups to effectuate immobilization of a biologic in a composition of the invention, to label a biologic with a probe or make other modifications to facilitate assays, or to introduce functionalities to effectuate binding of a biologic to a crosslinker. Methods for modifying biologics are known and described in, for example, U.S. Pat. Nos. 6,147,683 and 5,419,966; Cohen and Cech, J. Am. Chem. Soc., 119: 6259-6268 (1997); Protein Immobilization: Fundamentals and Applications, R. Taylor ed. (1991), see particularly chapters 3 and 10; and Immobilization of Enzymes and Cells, G. Bickerstaff ed. (1997). Some can also be obtained commercially (see, e.g., Operon Technologies, Inc. for custom oligonucleotide modifications such as amino modifications).

For example, for biopolymers that are peptides (e.g., proteins), such modifications are traditionally introduced by reacting targeted amino-acid residues of the peptides with an organic derivatizing agent that is capable of reacting with selected sides or terminal residues or by harnessing mechanisms of post-translational modifications that function in selected recombinant host cells. Such modifications are within the ordinary skill in the art and are performed without undue experimentation.

Cysteinyl residues most commonly are reacted with a-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(5-imidozoyl)propionic acid, chloroacetyl phosphate, N-alkylmaleimides, 3-nitro-2-pyridyldisulfide, methyl-2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful. The reaction is preferably performed in 0.1M sodium cacodylate at pH 6.0.

Lysinyl and amino-terminal residues can be reacted with succinic or other carboxylic acid anhydrides. Derivatization with these agents has the effect of reversing the charge of the lysinyl residues. Other suitable reagents for derivatizing α-amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate.

Arginyl residues can be modified by reaction with one or several conventional reagents, among them phenylglyoxal; 2,3-butanedione; 1,2-cyclohexanedione; and ninhydrin. Derivatization of arginine residues requires that the reaction be performed in alkaline conditions because of the high pKₐ of the guanidine functional group. Furthermore, these reagents can react with the groups of lysine as well as the arginine epsilon-amino group.

The specific modification of tyrosyl residues can be made, with particular interest in introducing spectral labels into tyrosyl residues, by reaction with aromatic diazonium compounds or tetranitromethane. N-acetylimidizole and tetranitromethane are most commonly used to form O-acetyl tyrosyl species and 3-nitro derivatives, respectively. Tyrosyl residues can be iodinated using ¹²⁵I or ¹³¹I to prepare labeled proteins for use in radioimmunoassay.

Carboxyl side groups (aspartyl or glutamyl) can be selectively modified by reaction with carbodiimides (R′—N═C═N—R′) such as 1-cyclohexyl-3-(2-morpholinyl-4-ethyl) carbodiimide or 1-ethyl 3-(4-azonia-4,4-dimethylpentyl) carbodiimide. Furthermore, aspartyl and glutamyl residues are converted to asparaginyl and glutaminyl residues by reaction with ammonium ions.

Glutaminyl and asparaginyl residues can be deamidated to the corresponding glutamyl and aspartyl residues. These residues can be alternatively deamidated under mildly acidic conditions. Either form of these residues falls within the scope of this invention.

Other modifications include hydroxylation of proline and lysine, phosphorylation of hydroxyl groups of seryl, threonyl, or tyrosyl residues, methylation of the a-amino groups of lysine, arginine, and histidine side chains (T. E. Creighton, Proteins: Structure and Molecular Properties, W. H. Freeman & Co., San Francisco, pp. 79-86 (1983)), acetylation of the N-terminal amine, and amidation of any C-terminal carboxyl group. The molecules can further be covalently linked to nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192; or 4,179,337.

Certain post-translational modifications can result from the action of recombinant host cells on the expressed polypeptide. Glutaminyl and aspariginyl residues are frequently post-translationally deamidated to the corresponding glutamyl and aspartyl residues. Alternatively, these residues can be deamidated under mildly acidic conditions. Either form of these residues falls within the scope of this invention.

Other post-translational modifications include hydroxylation of proline and lysine, phosphorylation of hydroxyl groups of seryl, threonyl, or tyrosyl residues, methylation of the a-amino groups of lysine, arginine, and histidine side chains (T. E. Creighton, Proteins: Structure and Molecular Properties, W. H. Freeman & Co., San Francisco, pp. 79-86 (1983)).

These modifications can also be useful for modifying cells. Cells can generally be covalently modified by derivatizing amino-acid side-chain groups of cell-surface proteins. For example, lysinyl and terminal amino residues of cell-surface proteins can be modified with amine-reactive groups (e.g., isocyanates and aldehydes) or crosslinked to other amines using a suitable reactive crosslinker (e.g., glutaraldehyde).

Chemical modification of nucleics can be more difficult to modify than peptides because groups found in peptides such as thiols, primary amines, and carboxylic acids are not abundant in naturally-occurring nucleics. But modification methods are known. For example, nucleics can be modified by reacting their cytidine residues with sodium bisulfite to form sulfonate intermediates, which can then be coupled to hydrazines or aliphatic amines (e.g., ethylenediamine). The amines thus formed are then able to react with a number of amine-reactive reagents as already described for proteins.

Terminal phosphate groups of nucleics can react with carboiimides and similar reagents in combination with nucleophiles to yield modified phosphodiesters, phosphoramidates, and phosphorothiates (see, e.g., Nucleic Acids Res., 22, 920 (1994)). For example, DNA can be reacted with carbonyl diimidazole and ethylenediamine to yield a phosphoroamide that has a primary amine, which can be modified with amine-reactive groups as already described to be within the scope of the invention. Modifications of hydroxyl groups of polysaccharides such as cellulose, agarose, and dextran are also within the scope of this invention and can be performed using cyanogen bromide (CNBr) in the presence of a strong base or by means of a suitable cyano-transfer reagent (e.g., triethylamine). The CNBr-activated saccharide yields a cyanate ester, which is able to react with nucleophilic compounds. Hydroxyl groups of polysaccharides can also be modified using 1,1′-carbonyldiimidazole (CDI) and subsequently reacted with amines. Additionally, polysaccharides can be treated with sodium periodate to yield polyaldehydes. Polyaldehydes can be further converted to polyamines by activation with ethylenediamine.

Water-Soluble Crosslinker

A composition of the invention also includes a water-soluble crosslinker. A crosslinker suitable for use in the invention is selected to provide a desirable gel time for a polyurethane-hydrogel composition of the invention and to provide a transparent composition.

The term “gel time” means the time that elapses between the time when a prepolymer and a crosslinker are first mixed together in aqueous solvent and the time at which that mixture polymerizes. Gel time can vary depending on the amount and type of hydrogel components selected as well as on the reaction conditions such as pH and temperature. The gel time is not critical for the composition and method of the invention. A suitable gel time will depend on the end-use application. That is, the gel time should be sufficiently long to allow for dispensing of the composition but not so long that the process for making the composition becomes prohibitively expensive or unfeasible for commercial purposes. For some applications, the gel time at 25° C. is generally less than about 5 minutes, preferably less than about 4 minutes, and more preferably less than about 2 minutes. And for some applications, such as high-throughput applications, a rapid gel time is preferred.

A crosslinker can be selected based on its functionality and water solubility. Generally a crosslinker should have a functionality (both number and type) that provides a reaction rate with the prepolymer that is at least 10 times faster than the reaction rate of water with the prepolymer. And the functionality preferably provides a reaction rate that is at least 100 times faster, more preferably at least 1,000 times faster, even more preferably at least 10,000 times faster, and still more preferably at least 100,000 times faster than the reaction rate of water with the prepolymer. The use of the term “prepolymer” in this paragraph refers to prepolymer as defined above as well as a prepolymer derivatized with a biologic.

A crosslinker generally has a functionality of at least 2, preferably at least 3, and more preferably at least 4, still more preferably at least 5, and even more preferably at least 6. But a crosslinker generally has a functionality of no more than about 40, preferably no more than about 30, and more preferably no more than about 20. In one embodiment, a crosslinker has a functionality of at least about 8 and no more than about 16. The term “functionality” is known to one of skill in the polyurethane art and generally refers to the number of atoms per molecule able to react with the unreacted isocyanate groups of the prepolymer. The functionality generally provides about 2 or more active hydrogen groups per molecule. The active hydrogen groups can be hydroxyl, mercaptyl, or amino groups.

In one embodiment, the functionality of the crosslinker can be modulated by the substrate. For example, a poly-L-lysine coated glass slide also provides isocyanate-reactive groups, and these isocyanate-reactive groups can contribute to the crosslinking of a composition of the invention.

The site or group (i.e., moles NCO×functionality/moles crosslinker×functionality) ratio of initial isocyanate to crosslinker is generally at least about 1.4, preferably at least about 1.6, and more preferably at least about 1.8. But this ratio is generally no greater than about 2.7, preferably no greater than about 2.4, and more preferably no greater than about 2.1.

The combination of functionality and water solubility of a crosslinker is selected to provide a polyurethane hydrogel of the invention with a desirable tensile modulus or number-average molecular weight between crosslinks and transparency.

It has surprisingly been found that the selection of the crosslinker is important to obtaining a transparent polyurethane hydrogel of the invention. Although this invention is not limited to any particular theory, it is believed that the selection of crosslinker according to the invention facilitates solubility of the polyurethane network as well as scavenges residual isocyanate. As a result, the crosslinker facilitates maintaining an aqueous phase and facilitates prevention of formation of an insoluble phase.

The term “residual isocyanate” means that amount of isocyanate that did not react in the formation of the prepolymer. That is, “residual isocyanate” means that amount of isocyanate that is still available for reaction after a prepolymer is formed. It is believed that residual isocyanate can react with water and contribute to formation of an insoluble phase, thereby adversely affecting transparency.

According to the invention, the site or group ratio of residual isocyanate to initial crosslinker functionality (i.e., moles NCO×functionality/moles crosslinker×functionality) is no greater than about 0.8, preferably no greater than about 0.7, and more preferably no greater than about 0.6. In one embodiment, this ratio is between about 0.4 and 0.5. In another embodiment, this ratio is about 0.47.

A crosslinker is present in an amount effective to form a network with the prepolymer and to scavenge or solubilize enough residual isocyanate to prevent formation of an insoluble phase, which substantially adversely affects transparency. A crosslinker should not be included in so large of an amount that it substantially adversely affects gel formation or segregate into a separate insoluble and nontransparent phase. The amount of crosslinker suitable for use with the invention will typically depend on the type of crosslinker selected and the prepolymer. One skilled in the art having read this specification would understand how to determine the amount of crosslinker suitable for the invention such that gelation occurs and the resulting polyurethane hydrogel has strength suitable for its end-use application.

In one embodiment, a crosslinker is solubilized in aqueous solvent, preferably water, to form a crosslinker solution. To control reactivity between a crosslinker and a prepolymer, the pH of the crosslinker solution can be controlled to promote reaction with prepolymer. For example, the pH of a 1 weight-percent solution of an amine-functionalized crosslinker (e.g., polyethylenimine) generally is at least about 7, preferably at least about 7.4, and more preferably at least about 7.8. But the pH generally is no greater than about 10, preferably no greater than about 8.6, and more preferably no greater than about 8.2. In one embodiment, the pH of a 1 weight-percent solution of an amine-functionalized crosslinker is about 8. The effective pH will depend on the type of crosslinker used. One skilled in the art having read this specification will recognize that any desirable pH control of the crosslinker solution will be unnecessary if a composition of the invention is prepared in an aqueous solvent that contains a pH buffer.

Examples of suitable crosslinkers include polyamines, amine end-capped polyols, polyols, and amine end-capped ethylene-oxide sugars.

Polyamines suitable for use with the invention have at least about 0.8 milliequivalent (meq) charge per gram of crosslinker. Suitable polyamines can have a charge density much higher than 0.8 meq charge per gram.

In one embodiment, a polyamine has 1.0 meq charge per gram, and in another embodiment, a polyamine has between 20 and 25 meq charge per gram. Suitable polyamines generally have a molecular weight of at least about 140 gram/mole, preferably at least about 170 gram/mole, and more preferably at least about 200 gram/mole. But suitable polyamines generally have a molecular weight no greater than about 2,000 gram/mole, preferably no greater than about 1,800 gram/mole, and more preferably no greater than 1,500 gram/mole.

In one embodiment, the polyamine is polyethylenimine having a molecular weight between about 600 gram/mole and about 800 gram/mole. Other molecular weights of polyethylenimine are also useful with the invention.

Polyols and amine end-capped polyols suitable for use with the invention are water soluble. Preferably they are ethylene-oxide based. Examples of polyamines include polyethylenimine (e.g., 600, 800, and 1200 molecular weight; e.g., CAS No. 25987-06-8), polyvinyl amine, and chitosan.

Although less preferred, other amine end-capped polyols include the water-soluble JEFFAMINE T-Series amines (e.g., JEFFAMINE T-403 [CAS 39423-51-3], which is a polyoxypropylenetriamine having an average molecular weight of approximately 440) and the JEFFAMINE ED-2003 amine [CAS 65605-36-9], which is a water-soluble aliphatic diamine derived from a propylene oxide-capped poly(ethylene oxide) with an approximate molecular weight of 2000 (trademark of, and available from, Huntsman, Ariz.). The functionality of an amine end-capped polyalkyleneoxide may be increased by initiating polymerization with a sugar (e.g., polyacrylic acid, sorbitol, sucrose, erythritol, and pentaerytheratol).

Examples of polyols include VORANOL RN-482 polyol (trademark of, and available from, The Dow Chemical Company) and VORANOL CP-450 polyol (trademark of, and available from, The Dow Chemical Company).

Water-soluble crosslinkers such as 3-, 4-, 5-, and higher functional amine end-capped polyethylene glycols should have sufficient molecular weight such that upon incorporation into a polyurethane hydrogel, the M_{c} is at least about 2,000, preferably at least about 3,000, more preferably at least about 4,000, and still more preferably at least about 5,000. But the M_{c} should be no greater than about 8,000, preferably no greater than about 7,000, and more preferably no greater than about 6,000.

In one embodiment, a crosslinker is also selected to substantially control the effect of nonspecific binding, e.g., nonspecific protein binding. Nonspecific binding includes any undesired disturbance in a signal derived from an analytical technique. Depending on the sensitivity of the activity technique, this disturbance is preferably controlled to provide an accurate signal. Nonspecific binding will be discussed in more detail below.

Examples of this type of crosslinker include multifunctional amine end-capped ethyleneoxides. One such crosslinker is shown here (I) and is a 4-functional poly(oxy-1,2-ethanediyl, α-hydroxy-ω-(2-aminoethoxy)-, ether with 2,2-bis(hydroxymethyl)-1,3-propanediol (4:1) (9C1) [CAS 169501-65-9], where n, which is the degree of polymerization in the structure below, can vary per chain but, in the end, typically corresponds to a final molecular weight of between about 484 and 1,189 grams/mole. Typically n can range from n=1 to n=5.

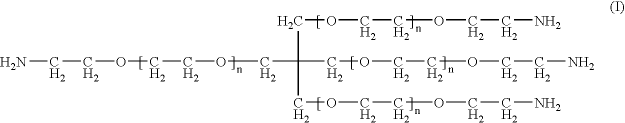

Other examples of amine end-capped poly(ethylene oxide) crosslinkers having a functionality of 2 to 12 can be found in the literature and include CAS Registry numbers 177986-99-1P; 179189-24-3; 52379-15-4; 244235-34-5; 244235-35-6; 244235-36-7; 244235-38-9; 172355-14-5; 180273-44-3; and 158948-29-9. Mono-, di-, and multifunctional polyalkylene oxides including poly(ethylene oxide) or polyethylene glycol are commercially available from Shearwater Polymers, Inc. (Huntsville, Ala.). One skilled in the art having read this specification can easily imagine derivatives of these crosslinkers that would also be useful for the invention and such derivatives are considered to be within the scope of this invention. These types of compounds have been described in, for example, Urrutigoity and Souppe, Biocatalysis, 2:145 (1989); Cordes and Kula, J. Chromat., 376:375 (1986); and Okada and Urabe, Meth. Enzymol., 136:34 (1987) for uses other than as described for this invention, but they have surprisingly been found to be useful for this invention both for material properties and for biocompatibility properties.

One advantage of the water-soluble crosslinkers useful according to this invention includes their ability to react with immobilizing agents through active-hydrogen groups after a composition of the invention is polymerized. This can be useful to immobilize a biologic in a composition of the invention after the composition has polymerized.

One skilled in the art having read this specification will readily be able to select the type and amount of water-soluble crosslinker useful according to the invention. In making such a selection, Applicants have surprisingly found that this selection can be optimized for end-use applications that can be affected by activity measured from nonspecific binding, such as assays useful for diagnostic devices and therapeutic applications. For such end-use applications, activity from nonspecific binding can be problematic. Nonspecific binding includes any binding of a probe to a polyurethane hydrogel that subsequently provides activity for a false positive or activity that falsely enhances a positive signal. This can also be known as noise and can be monitored by a signal-to-noise ratio. Many of the Examples in this specification use the water-soluble crosslinker polyethylenimine (molecular weight of 700) at a final concentration of 0.1% (w/v). This water-soluble crosslinker is useful for the end-use applications described in this specification. But it may be desirable to reduce the activity measured from nonspecific binding relative to the polyethylenimine crosslinker (0.1 % (w/v)). This can be done by, for example, optimizing the amount of crosslinker used to prepare a polyurethane hydrogel, selecting alternative crosslinkers such as those already mentioned above, treating crosslinkers or a polyurethane hydrogel with blocking agents, or a combination of these.

Another option directed to reducing activity measured from nonspecific binding includes blocking active-hydrogen groups available on the crosslinker by, for example, capping active-hydrogen groups. This can be accomplished by contacting the polyurethane hydrogel having an immobilized biologic with a blocking agent or by contacting a water-soluble crosslinker with a blocking agent to form a treated crosslinker and then admixing the treated crosslinker with appropriate hydrogel components. Blocking agents are known to one of skill in the art and readily commercially available. One example of a blocking agent includes acetic anhydride, which can cap amine groups. Generally a polyurethane hydrogel is contacted with a blocking agent after a biologic is immobilized in a polyurethane hydrogel to avoid substantial interference of immobilization of the biologic in the polyurethane hydrogel.

This effect on activity measured from nonspecific binding is surprising and is advantageous for biomedical applications according to the invention.

Additives

A composition of the invention can also include known additives and other known components to prepare a polyurethane-hydrogel composition. Generally any additive or combination of additives known to one of skill in the art to be useful in preparing a polyurethane-hydrogel composition, particularly protein-stabilizing additives, can be included in a composition of the invention so long as the additive or combination of additives is not substantially incompatible with other components in the composition and so long as the additive or combination of additives does not substantially adversely affect the transparency of the composition or the immobilization of the biologic in the composition.

Examples of protein-stabilizing additives include antioxidants, preservatives, antifreeze, chelators, lyoprotectants, surfactants, and other additives suitable for maintaining or stabilizing the activity of a protein.

Antioxidants suitable for use according to the invention are effective to retard free-radical degradation of the composition or biopolymer and include vitamin E, vitamin C, and butylated hydroxytoluene.

Preservatives suitable for use according to the invention are effective to retard or prevent microbial proliferation in a composition of the invention and include octadecyldimethylbenzyl ammonium chloride, benzalkonium chloride (a mixture of alkylbenzyldimethlyammonium chlorides in which the alkyl groups are long-chain compounds), and benzethonium chloride. Other types of preservatives include aromatic alcohols such as phenol, butyl and benzyl alcohol, allyl parabens such as methyl or propyl paraben, catechol, resorcinol, cyclohexanol, 3-pentanol, m-cresol, glutaraldehyde, and azide.

Antifreeze suitable for use according to the invention is effective to facilitate freeze stability of a composition of the invention and includes methanol, ethanol, ethylene glycol, glycerol, polyethylene glycol, and isopropyl alcohol.

Lyoprotectants suitable for use according to the invention are effective to reduce or prevent chemical or physical instability of a protein upon lyophilization and storage. Examples of suitable lyoprotectants include sugars such as sucrose or trehalose; an amino acid such as monosodium glutamate or histidine; a methylamine such as betaine; a lyotropic salt such as magnesium sulfate; a polyol such as trihydric or higher sugar alcohol (e.g., glycerin, erythritol, glycerol, arabitol, xylitol, sorbitol, and mannitol); propylene glycol; polyethylene glycol; and PLURONICS surfactants (trademark of, and available from BASF).

Chelators suitable for use according to the invention are effective to bind metals that may interfere with desired activity. Examples of chelators include ethylenediamine tetraacetic acid (EDTA); [(ethylenedioxy)diethylenedinitrolo]tetraacetic acid (EGTA); 1,10-phenanthroline; pyridine-2,6-dicarboxylic acid (dipicolinic acid); and 8-hydroxyquinoline (oxine).

Surfactants can also be desirable additives to retard aggregation of some biopolymers. Examples of suitable surfactants include nonionic surfactants such as polysorbates (e.g., polysorbates 20 or 80); poloxamers (e.g., poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl-, or stearyl-sarcosine; linoleyl-, myristyl-, or cetyl-betaine; lauroamidopropyl-, cocamidopropyl-, linoleamidopropyl-, myristamidopropyl-, palnidopropyl-, or isostearamidopropyl-betaine (e.g lauroamidopropyl); myristamidopropyl-, palmidopropyl-, or isostearamidopropyl-dimethylamine; sodium methyl cocoyl-, or disodium methyl oleyl-taurate; and the MONAQUAT™ surfactants (Mona Industries, Inc., Paterson, N.J.); polyethyl glycol, polypropyl glycol, and copolymers of ethylene and propylene glycol (e.g., Pluronics, PF68).

Desirable additive selection and amount can be determined for each combination of additive and biologic based on known methods without undue experimentation, and one skilled in the art having read this specification can apply known methods to select appropriate additives and amounts (see, e.g., Methods in Enzymology, Vol. 182: Guide to Protein Purification, M. P. Deutscher ed. (1990), particularly the chapter directed to General Methods for Handling Proteins and Enzymes).

The balance of a composition of the invention is aqueous solvent. Any aqueous solvent or combination of aqueous solvents that does not substantially adversely affect immobilization of a water-soluble biopolymer in a polyurethane-hydrogel composition of the invention can be used. Examples include water and sterile water; any water-containing solvent such as sterile saline solution, Ringer's solution, dextrose solution, and pH buffer; and a combination of aqueous solvents. The type and amount of the aqueous solvent selected can depend on the biopolymer selected and the end-use application. This selection is within the knowledge of one skilled in the art having read this specification. A composition of the invention generally includes aqueous solvent in an amount effective to disperse hydrogel components.

Buffers are particularly useful for preserving activity of biopolymers selected from peptides and compounds containing amino acids such as proteins in a composition of the invention. Examples of suitable pH buffers include histidine buffer, potassium-phosphate buffer, tris buffer, succinate buffer, citrate buffer, acetate buffer, MOPS buffer (3-(N-Morpholino)propanesulfonic acid), HEPES buffer (N-2-hydroxyethylpiperazine-N′-2-ethanesulfonic acid), and TEA (triethanolamine). A desirable pH for preparing a composition of the invention generally depends on the reactivity of the isocyanate with the isocyanate-reactive groups of the crosslinker. But this pH should not be so high or so low that the biologic irreversibly denatures. For many biologics, including cells and proteins, the pH to which they are exposed should be at least about 5, preferably at least bout 6, and more preferably at least about 6.5. But the pH should be no more than about 9, preferably no more than about 8, and more preferably no more than about 7.5. One skilled in the art will appreciate that once a composition of the invention is polymerized, it can be appropriate to, for example, wash the polyurethane hydrogel or perform a buffer exchange with the polyurethane hydrogel to optimize the pH environment for immobilized biologics to, for example, optimize activity.

Such compositions of the invention can be desirably prepared using buffer concentrations of at least about 10 millimolar (mmolar), preferably at least about 30 mmolar, and more preferably at least about 40 mmolar. Generally these buffer concentrations are no greater than about 200 mmolar, preferably no greater than about 80 mmolar, and more preferably no greater than about 60 mmolar. One of skill in the art will readily recognize that a desirable buffer concentration will depend on the biologic immobilized.

In one embodiment, a composition of the invention is storage stable. A storage-stable composition is one in which a biologic immobilized in the composition essentially maintains detectable activity upon storage. Stability can be measured at a selected temperature for a selected period of time.

In one embodiment, a composition of the invention having a biologic selected from a peptide, particularly a protein, can be storage stable at room temperature for at least about 2 hours, preferably at least about 1 day, more preferably at least about 3 days, even more preferably at least about 7 days, and still more preferably at least about 14 days. Such a composition of the invention can alternatively be stable at 2-4° C. for at least about 2 days, preferably at least about 6 days, more preferably at least about 14 days, even more preferably at least about 21 days, and still more preferably at least about 30 days.

In another embodiment, a composition of the invention is treated such that the biologic is dehydrated after the composition has polymerized. Preferably the biologic is a peptide, particularly a protein. Dehydration can be accomplished using methods known to one of skill in the art (see, e.g., Stability and Stabilization of Biocatalysts, A. Ballesteros et al. eds. (1998) and particularly the chapter directed to Some Factors Affecting the Behavior of Anhydrous α-Chymotrypsin at High Temperature at p. 59).

The dehydrated biologic immobilized in a composition of the invention can subsequently be rehydrated with an aqueous solvent. Reconstitution can be accomplished by, for example, immersing the composition in an aqueous solvent or washing the composition with an aqueous solvent.

Preferably a composition of the invention having a dehydrated biologic that is a peptide, particularly a protein, is storage stable such that it can be storage stable at room temperature for at least about 4 days, preferably at least about 12 days, more preferably at least about 20 days, and still more preferably at least about 30 days, where the total number of days refers to the time period the biologic is dehydrated with the measurement for activity being determined near in time to, preferably immediately subsequent to, rehydration of the biologic.

In yet another embodiment, a protein immobilized in a polyurethane hydrogel has enhanced stability when compared to the same protein free in solution or suspension. That is, the immobilized protein loses activity over time at a slower rate than does the free protein. And, over time, the free protein may show no detectable activity while the immobilized protein still shows detectable activity. One such example is shown in Example 7.

Articles of manufacture

A composition of the invention can be applied to a substrate suitable for storage or transportation of the composition or for an intended end-use application. An article of manufacture includes a substrate having a polyurethane hydrogel with a biologic immobilized in the polyurethane hydrogel and includes a substrate having a polyurethane hydrogel suitable for subsequently immobilizing a biologic in the polyurethane hydrogel. Any amount of a composition of the invention that is suitable for the intended end-use application can be applied to the substrate. For some biomedical applications, an amount great enough to provide a film that is 20 to 200 μm (micrometers) thick is desirable.

A suitable substrate for holding a composition of the invention includes any substrate that does not substantially adversely affect the intended end-use application of the substrate having a composition of the invention and that is not substantially adversely affected by any solution to which the substrate will be exposed for the intended end-use application. Examples of suitable substrates include a microporous or nonwoven membrane, particulate porous or nonpourous media, or a nonporous device such as a microscope slide or a microtiter plate.

Microporous materials include membranes prepared from nylon, polypropylene, polyesters, polyvinyl fluoride, Teflon (trademark of E.I. DuPont de Nemours & Co.), or cellulose. Membranes of woven or nonwoven materials may be of suitable surface area such that any test fluid containing a prospective biospecific agent will wet the surface and may or may not pass through the membrane. Membranes with pore sizes of about 0.05 to about 5.0 microns are typically used. The membrane should be substantially compatible with the composition of the invention as well as any solution to which the membrane will be subjected.

Particulate porous or nonporous media include inorganic particles such as silica gel and organic particles such as charcoal, polystyrene, and polyamine particles. The particle size will generally be selected based on the intended end-use application of the support and composition.

Nonporous devices include a microscope slide, microtiter plate, and other assay devices. These types of devices are generally prepared from glass, polystyrene, polypropylene, and polyvinylchloride and are generally commercially available.

For some substrates, a substrate is coated with a coating compound suitable for facilitating the interaction of the composition of the invention with the substrate so the composition can adhere to the substrate. A coating compound includes any compound that can react, either ionically or covalently, with at least the surface of the substrate and with at least some portion of the composition of the invention to adhere to the substrate. The term “adhere” means that the composition of the invention is sufficiently attached to the substrate so it is suitable for its end-use application. For example, a glass substrate can be coated with an amine (e.g., alkyl amines such as lysine and polylysine and aryl amines) to facilitate adherence of the composition to the substrate. The use and selection of coating compounds are known to one of skill in the art and described in, for example, Molecular Cloning: A Laboratory Manual, Sambrook and Russel eds. (2000).

A substrate can generally be in any form or shape suitable for an intended end-use application such as particles, plates, wells, films, beads, and tapes.

An article of manufacture can also be a kit and include other materials desirable from a commercial or end-user standpoint. Such materials include aqueous solvent to, for example, hydrate a dehydrated peptide, particularly a protein, immobilized on a composition of the invention; reagents suitable for detection assays such as fluorescent tags, dyes, or other protein-staining compounds, and other detection components; and instructions for use.

Method

One method of the invention includes immobilizing a biologic in a polyurethane-hydrogel composition. Another method of the invention includes preparing a polyurethane-hydrogel composition having a biologic immobilized in the composition.

A method of the invention includes admixing a prepolymer, a biologic, and a water-soluble crosslinker in an aqueous solvent and in the substantial absence of organic solvent. According to the invention, the prepolymer, the water-soluble crosslinker, or a combination of these can first be derivatized and then polymerized with the appropriate hydrogel components in a stepwise fashion. Alternatively, the prepolymer, the water-soluble crosslinker, or a combination of these can be derivatized with a biologic and polymerized with the appropriate hydrogel components substantially concurrently. According to the invention, the method is carried out substantially free of organic solvent.

Additives can be included in a composition of the invention during any step of the method. For example, an additive can be dispersed with a prepolymer in aqueous solvent to form a prepolymer solution, which can be subsequently admixed with a biologic and a water-soluble crosslinker. As another example, an additive can be dispersed with a biologic to form a biologic solution, which can be subsequently admixed with a water-soluble crosslinker and a prepolymer. As yet another example, an additive can be dispersed with a biologic and a water-soluble crosslinker to form a biopolymer/crosslinker solution, which can be subsequently admixed with a prepolymer to form a composition of the invention.

The conditions are generally selected such that they are not substantially incompatible with hydrogel components or with immobilization of a biologic in a polyurethane-hydrogel composition. These conditions can be selected without undue experimentation by one skilled in the art having read this specification. These conditions, for example, temperature, pH, buffer concentration, and mixing, will vary depending on the biologic selected.

Also according to the invention, the reaction mixture can be deposited onto a substrate during any step. For example, after the stepwise method or the concurrent method, the reaction mixture can be deposited onto a substrate. Alternatively, the derivatized prepolymer can be deposited onto a substrate and then polymerized with crosslinker, or the prepolymer can be deposited onto a substrate and then derivatized and polymerized on the substrate. As another alternative, a crosslinker can be derivatized with a biologic and deposited onto a substrate and then polymerized with a prepolymer.

A method of the invention also includes preparing a polyurethane-hydrogel composition by admixing a prepolymer and a water-soluble crosslinker in an aqueous solvent but in the substantial absence of organic solvent and then immobilizing a biologic in the composition by contacting the composition with the biologic. For example, a composition can be prepared and dispensed onto a substrate and then subsequently contacted with a biologic. As an alternative example, a composition can be prepared, dispensed onto a substrate, polymerized into a polyurethane hydrogel, and then contacted with a biologic either immediately or at anytime after, for example, shipment or storage of the polyurethane hydrogel. When contacting a polymerized polyurethane-hydrogel composition with a biologic, it may be preferred to contact the polyurethane hydrogel with an immobilizing agent suitable for subsequently interacting with the biologic to immobilize the biologic in the polyurethane hydrogel or to derivatize the biologic with an immobilizing agent and subsequently contact the polyurethane hydrogel with the derivatized biologic.

The following description provides one example of a method of the invention. To prepare a polyurethane-hydrogel composition of the invention, a prepolymer can be dispersed in aqueous solvent. One skilled in the art having read this specification would understand that conventional mixing methods can be used to disperse the prepolymer in aqueous solvent. The prepolymer can then be derivatized with a biologic. Derivatization generally occurs by covalently reacting isocyanate groups of the prepolymer with isocyanate-reactive groups of the biomolecule or, if an immobilizing agent is used, by covalently linking a prepolymer and a biologic via an immobilizing agent. Some derivitization may also occur by other interactions with the network of the polyurethane hydrogel.

Next, a crosslinker solution can be added to the prepolymer, and the mixture can be stirred for an amount of time effective to disperse hydrogel components in aqueous solvent. Again, one skilled in the art having read this specification would understand that conventional mixing methods can be used.

The composition can then be deposited onto a substrate suitable for an intended end-use application.

Biomedical Applications

A composition of the invention is particularly useful in biomedical applications. The term “biomedical application” includes any research or medical application in which selective interaction between a biologic and a biospecific agent is desirable.

Examples of biomedical applications include assays useful for diagnostic devices and therapeutic applications.

Diagnostic devices include devices suitable for diagnosing disease or detecting the presence of particular biospecific agents. Diagnostic devices can include test strips, protein arrays and microarrays, DNA arrays and microarrays, and cell arrays and microarrays. Protein arrays and microarrays are diagnostic devices that are known and described in, for example, Global Analysis of Protein Activities Using Proteome Chips, SCIENCE, 293: 2101-2105 (2001), Printing Proteins as Microarrays for High-Throughput Function Determination, SCIENCE, 289: 1760-1763 (2000), and Protein Microarrays: Prospects and Problems, CHEMISTRY AND BIOLOGY, 872:105-115 (2001). DNA arrays and microarrays are also known and described in, for example, Molecular Cloning: A Laboratory Manual, Sambrook and Russel eds. (2000). Cell arrays are also known and described in, for example, Genomic Advances, CHEMICAL & ENGINEERING NEWS, pp. 43-57, 9 Jul. 2001 and Microarrays of Cells Expressing Defined cDNAs, NATURE, 411:107-110 (2001).

Methods for DNA and protein microarray fabrication include contact printing (see, e.g., Microarrays: Biotechnology's Discovery Platform for Functional Genomics, TIBTECYH, 16: 301-306 (1998) and Quantitative Monitoring of Gene Expression Patterns with Complementary DNA Microarrays, SCIENCE 270: 467-470 (1995)), photolithography (see, e.g., Massively Parallel Genomics, SCIENCE, 277: 393-395 (1997)), soft lithography (see, e.g., Soft Lithography, ANGEW. CHEM. INT. ED., 37: 550-575 (1998)), ink-jet printing (see, e.g., Microarrays: Biotechnology's Discovery Platform for Functional Genomics, TIBTECYH, 16: 301-306 (1998) and Expression Profiling Using Microarrays Fabricated by an Ink-jet Oligonucleotide Synthesizer, NAT. BIOTECH, 19: 342-347 (2001)), bubble-jet printing (see, e.g., Microarray Fabrication with Covalent Attachment of DNA Using Bubble-jet Technology, NAT. BIOTECH. 18: 438-441 (2000)), and piezoelectric printing (see, e.g., Piezoelectric Arrays, NAT. BIOTECH., 19: 739 (2001)).

Therapeutic applications include applications suitable for delivering medical treatment to, for example, a mammal, particularly a human but also animals such as a dog, a cat, a horse, or a monkey.

Assays include any test suitable for quantitatively or qualitatively determining the activity, potency, strength, hybridization, expression, or other biological property of a biospecific agent or for quantitatively or qualitatively determining the presence of a biospecific agent in a mixture or test sample. Assays also include screens, which include any technique suitable for sifting data for selection of a particular phenomenon or result. Screens can include affinity matrices.

Assays can be performed in a variety of formats, including protein-protein binding assays, biochemical screening assays, and immunoassays. These assays can involve direct detection such as by colorimetric analysis or label analysis (e.g., measure radioactivity, luminescence, optical density, or electron density) or indirect detection such as an epitope tag. A variety of assays suitable for biologics are known and described in, for example, Methods of Enzymatic Analysis 3^{rd} ed., Berg Meyer et al. (1983); Enzyme Assays: A Practical Approach, R. Eisenthal and M. Danson eds. (1993); and Manual of Industrial Microbiology and Biotechnology 2^{nd} ed., Denain et al. eds. (1999) (see chapter 13, which is particularly useful for describing cell reactions and monitoring cell reactions).

One example of a suitable assay includes an assay for identifying lead compounds for therapeutically-active agents that modulate binding of a biologic to its complementary biospecific agent. Another example of a suitable assay includes an assay for identifying lead compounds that mimic the biological activity of a native biospecific agent that is complementary to a biologic.

An assay according to the invention can be amenable to high-throughput screening of chemical libraries and is particularly useful for identifying small-molecule drug candidates.

A composition of the invention is particularly useful for protein-microarray applications. A protein-microarray application is an assay that can accommodate low sample volumes (e.g., about 50 microliters) but allow for parallel analysis of many proteins (e.g., hundreds to thousands). In a protein-microarray application, a protein is immobilized in a composition of the invention as already described and various protein properties are evaluated. The evaluation includes screening for protein-protein interactions, identifying protein substrates, and identifying interactions with small molecules.

One type of useful microarray includes protein-function microarrays. A protein-function microarray can includes thousands of samples having a composition of the invention (each composition having a different protein immobilized in the composition) in a defined pattern. This microarray allows for massively parallel or high-throughput testing of a protein function. For example, the microarray can be contacted with a probe sample containing a fluorescently-labeled biospecific agent (e.g., protein, ligand, and small molecule). Any immobilized protein that tests positive for fluorescence is considered a candidate for binding to the biospecific agent. A probe sample includes any sample that is selected to contact a polyurethane hydrogel having a biologic immobilized in a polyurethane hydrogel. A probe sample can include any sample that can include a prospective biospecific agent such as a crude or heterogeneous sample (e.g., blood sample, bodily fluid sample, or tissue sample) or a pure or homogeneous sample (e.g., purified biospecific agent in aqueous solvent). The probe samples can, but need not, include detection labels.

One skilled in the art will appreciate that a biologic immobilized in a polyurethane hydrogel can contain a detection label or a component of a probe sample can contain a detection label. But it is not required that either the immobilized biologic or the probe sample contain a detection label so long as a detection component can be introduced by an assay. Assay methods for crude probe samples or for probe samples free of a detection label are known and described in, for example, Baird et al., Current and Emerging Commercial Optical Biosensors, J. Mol. Recognition, 14: 261-268 (2001); Rich et al., Survey of the Year 2000 Commercial Optical Biosensor Literature, J. Mol. Recognition, 14: 273-294 (2001); and Kodadek, Protein Microarrays: Prospects and Problems, Chem. and Biol., 8: 105-115 (2001).

A composition of the invention is particularly useful for cell-microarray applications. In such an application, a cell is immobilized in a composition of the invention as already described, and the composition is dispensed onto a substrate.

In one embodiment of a cell-microarray application, a cell capable of expressing a specific protein is immobilized in a composition. For this capability, a cDNA for a specific protein is incorporated into an expression vector. A transfection reagent is added to the cells, and then the cells are immobilized and induced to produce protein.

In another embodiment, a cell is first immobilized in a composition, the composition is dispensed onto a substrate, and then the cells are exposed to the expression vector and transfection reagent.

A cell microarray can be useful to conduct cell-based functional arrays and to monitor protein production (see, e.g., Manual of Industrial Microbiology and Biotechnology 2^{nd} ed., Denain et al. eds. (1999) (see chapter 13, which is particularly useful for describing cell reactions and monitoring cell reactions).

The invention will be further described by the following Examples. These Examples are not meant to limit this invention but to further illustrate embodiments of the invention. Numbers or letters in parentheses after samples refer to the corresponding number or letter shown in the Figure cited in each Example.

### EXAMPLES

### Example 1

### Preparation of Prepolymer Suitable for Use in the Invention

To prepare one example of a prepolymer suitable for use with the invention, a 7000 molecular-weight triol copolymer of ethylene oxide (75%) and propylene oxide (25%) (PLURACOL 1123™ available from BASF, Mount Olive, N.J.) (“the polyol”) was dried. Phosphoric acid (20 ppm) was added to the polyol. Next, the polyol (1687.46 g) was mixed with 165.0 g isophorone diisocyanate (IPDI) (available from Bayer, Pittsburgh, Pa.) and heated at 70° C. under dry nitrogen. Isocyanate levels were determined by addition of dibutylamine and back titration with standard acid. Fourteen days were required for the isocyanate concentration to reach 0.47 meq/g (0.39 meq/g=theoretical) according to ASTM No. D5155-96. The resulting prepolymer was liquid at room temperature (25° C.). This prepolymer is available from The Dow Chemical Company (HYPOL G-50 hydrophilic polymer).

### Example 2

### Immobilization of Human Serum Albumin in a Polyurethane-Hydrogel Composition

To immobilize human serum albumin in a polyurethane-hydrogel composition of the invention, human serum albumin (A4327, available from Sigma, St. Louis, Mo.) was immobilized on the prepolymer of Example 1. Polymerization of the prepolymer was initiated concurrently with the immobilization, and the polymerization mixture was deposited onto a glass microscope slide.

The following steps were taken to immobilize human serum albumin in the prepolymer and to polymerize the prepolymer. A first solution having human serum albumin and a crosslinker was prepared. To make this solution, a 1% (weight/volume) aqueous solution of low molecular-weight polyethylenimine (molecular weight of approximately 700) (PEI) (catalog no. 40,871-9, available from Aldrich, Milwaukee, Wis.) was first prepared and buffered to a pH of 7.8 using 18 molar sulfuric acid.

Human serum albumin was then dissolved to a concentration of 10 mg/ml in the polyethylene-imine solution to make a biopolymer solution.

A prepolymer solution was also prepared. To make this solution, the prepolymer of Example 1 was dissolved in distilled water by rapid mixing to make a concentration of 2.5% (weight/volume). The prepolymer solution (9 parts) was mixed with the biopolymer solution (1 part) within 5 minutes of making the prepolymer solution by rapid mixing to form a polymerization mixture. About 100 μL (microliters) of the polymerization mixture were aliquoted onto a microscope slide, which was precoated with polylysine. The mixture rested for about 2 hours under room temperature and standard pressure to form a polyurethane-hydrogel composition. Once the hydrogel was polymerized, the microscope slide was washed with phosphate-buffered saline (PBS) (137 mM NaCl, 2.7 mM KCl, 10 mM Na₂HPO₄, and 1.8 mM KH₂PO₄) at pH 7.4 to remove any unbound human serum albumin, prepolymer, or polyethylenimine. This washing step was carried out according to standard washing techniques known to one of skill in the art. In general, the microscope slide having the polyurethane-hydrogel composition was put into a plastic weigh boat and immersed in PBS. The weigh boat was placed on a rocking shaker and gently agitated for about 24 hours. The PBS was changed about every 3 to 4 hours.

As a control, a polyurethane-hydrogel composition was prepared as described above, except the biopolymer solution contained only PEI and was free of human serum albumin.

The control composition and the composition containing human serum albumin were then probed using a polyclonal goat antihuman-albumin antibody (A-1151, available from Sigma, St. Louis, Mo.) that was covalently conjugated with a fluorescent tag (6-(((4,4-difluoro-5-(2-thienyl)-4-bora-3a,4a-diaza-s-indacene-3-yl)styryloxy)acetyl)aminohexanoic acid, succinimidyl ester (BIODIPY 630/650-X,SE) (available from Molecular Probes, Eugene, Oreg.) through an activated succinimdyl functionality. This conjugation can be carried out using standard methods known to one of skill in the art (see, e.g., Brecher et al., TRANSFUSION, 40(4):411-413 (2000)). To analyze the compositions using this probe, the probe was diluted in an aqueous solution of PBS at pH 7.4 to a concentration of 0.5 μg/ml. The probe solution was added to each microscope slide by immersing the slide in the probe solution and gently agitating the probe solution for about 1 hour by using a rocking shaker. The probe solution was then poured off and the slide was washed as described above except that it was washed for about 48 hours and the buffer was changed every 8 to 10 hours.

The fluorescence of each composition was then determined by exposing each microscope slide to a Typhoon 8600 Fluorescence scanner (available from Molecular Dynamics, Sunnyvale, Calif.).

The composition containing human serum albumin showed a relative fluorescence signal greater than that of the control composition.

These results show that the human serum albumin maintained its biological activity—i.e., its physical stability—upon immobilization in the polyurethane-hydrogel composition such that the antibody recognized its antigen (human serum albumin).

### Example 3

### Evaluation of Polymerization Conditions

A polyurethane-hydrogel composition of the invention preferably polymerizes in a time period sufficiently long to allow for dispensing of the composition (e.g., dispensing onto a substrate) but not so long that a process for making the composition becomes prohibitively expensive or ineffective for making the composition. Polymerization time can be controlled by selection of type and amount of the components included in the composition or condition such as prepolymer amount and type, crosslinker amount and type, and pH.

To determine the effect of prepolymer and pH selection, polyurethane-hydrogel compositions having human serum albumin immobilized in the composition were prepared using varying amounts of prepolymer and varying pH.

A biopolymer solution was prepared according to Example 2, except that the polyethylenimine was present in an amount of 0.1% (weight/volume) and the human serum albumin was present in an amount of 0.1% (weight/volume) and the solution was adjusted to a pH as shown in Table 1 by adding 18 molar sulfuric acid.

A prepolymer solution was also prepared according to Example 2, except that the concentration was modified as described in Table 1.

The prepolymer solution (9 parts) was rapidly mixed with the biopolymer solution (1 part) within 5 minutes of making the prepolymer solution to form a polymerization mixture, which was aliquoted (100 μl) onto a slide coated with polylysine positioned substantially perpendicular to gravity. The amount of time needed for the polymerization mixture to gel was recorded. The polymerization mixture was considered to be gelled when it no longer flowed from its own weight—i.e., the mixture did not flow when the slide was tipped to be substantially parallel with the force gravity.

TABLE 1
Polymerization Conditions of a Composition According to the Invention

| Prepolymer Solution | | Polymerization Mixture |
|---|---|---|
| Prepolymer Concentration | | Gel Time |
| % (weight/volume) | pH | (Minutes) |
| 2.0 | 7.8 | No polymerization after 60 |
| 2.0 | 8.0 | No polymerization after 60 |
| 2.0 | 8.2 | No polymerization after 60 |
| 2.5 | 7.8 | 30 |
| 2.5 | 8.0 | 20 |
| 2.5 | 8.2 | 15 |
| 3.0 | 7.8 | 15 |
| 3.0 | 8.0 | <5 |
| 3.0 | 8.2 | <5 |

These results show that gel time can be affected by selection of prepolymer concentration and pH.

### Example 4

### Immobilization of Lactate Dehydrogenase in a Polyurethane-Hydrogel Composition

To study the stability of an enzyme that is immobilized in a polyurethane-hydrogel composition, the enzyme lactate dehydrogenase was immobilized in a polyurethane-hydrogel composition, and lactate dehydrogenase was tested for retention of activity after immobilization.

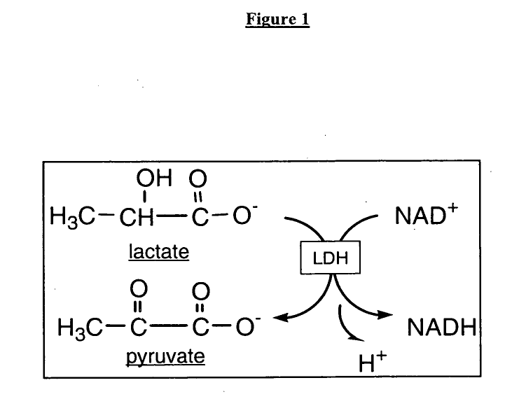

Lactate dehydrogenase (LDH) catalyzes the oxidation of lactate to pyruvate with concomitant reduction of the coenzyme NAD⁺ (nicotinamide adenine dinucleotide) to NADH. The reaction scheme is shown in FIG. 1.

To immobilize the enzyme in a polyurethane-hydrogel composition, the following steps were taken.

A biopolymer solution having the enzyme, the coenzyme, and a crosslinker was prepared. To make this solution, 1 U/ml lactate dehydrogenase (Sigma, Milwaukee, Wis.; catalog number L-1254), 10 mM NAD⁺ (Sigma; catalog number N-6522), and 1% (w/v) low MW polyethylenimine (Aldrich, Milwaukee, Wis.; catalog number 40,871-9) [polyethylenimine was adjusted to pH=8.0 using 18 M H₂SO₄] were mixed.

A prepolymer solution was prepared as described in Example 2.

The prepolymer solution (9 parts) and the biopolymer solution (1 part) were mixed thoroughly within 5 minutes of making the prepolymer solution to form a polymerization mixture. The polymerization mixture was dispensed into a 96-well microtiter plate (300 μl/well). The mixture was left to polymerize for at least 1 hour at room temperature and standard pressure. The mixture was then sealed with parafilm and covered to prevent drying.

The enzyme was analyzed for activity at various time points at room temperature by adding 3 mM lactate substrate (Sigma L-7022) and measuring the rate of NADH formation by monitoring fluorescence (excitation=λ₁340 nm; emission=λ₂465 nm). Fluorescence was monitored using a SpectraMax GeminiXS microplate fluorescence reader equipped with SOFTmax®Pro version 3.1 software (software and reader available from Molecular Devices Corporation, Sunnyvale, Calif.). The rate was measured in relative fluorescence units per second (rfu/sec).

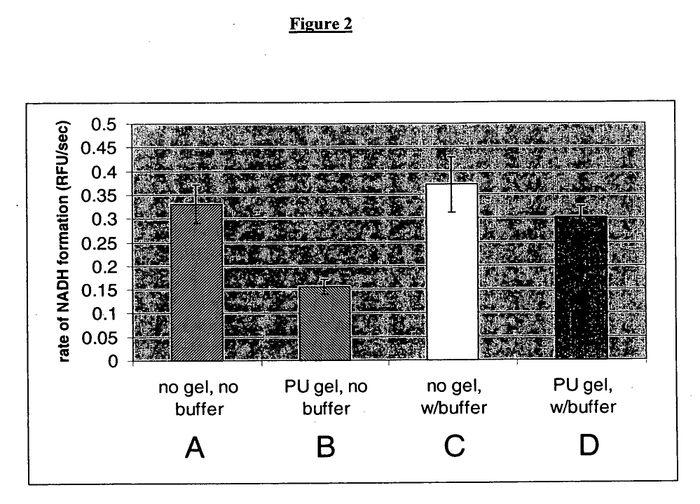

This analysis was carried out for enzyme immobilized in a polyurethane-hydrogel composition as well as for free enzyme in the biopolymer solution (that is, free in solution and not immobilized in a polyurethane-hydrogel composition). The results from this analysis are shown in FIG. 2. FIG. 2 shows that free enzyme (Sample A) and immobilized enzyme (Sample B) were both active and that immobilized enzyme was about 47% as active as free enzyme. These results indicate that NAD⁺ was maintained within the network of the gel.

### Example 5

### Effect of Buffer on Lactate Dehydrogenase Immobilized in a Polyurethane-Hydrogel Composition

To determine the effect of buffer on an enzyme immobilized in a polyurethane-hydrogel composition, lactate dehydrogenase was immobilized in a polyurethane-hydrogel composition as described in Example 4, except that the prepolymer solution was modified as described below.

The prepolymer solution was prepared by dissolving the prepolymer in 50 mM potassium-phosphate buffer having a pH of 8.0 (pH adjusted with 6 M potassium hydroxide) instead of distilled water.

The enzyme was analyzed for activity as described in Example 4. FIG. 2 shows that free enzyme (Sample C) and immobilized enzyme (Sample D) were both active and that immobilized enzyme was about 81% as active as free enzyme. The activity of the free enzyme for this analysis was determined for the enzyme in a biopolymer solution as described in Example 4, except that the solution also included 50 mM potassium phosphate at pH 8. These results also suggest that the activity of an enzyme immobilized on a polyurethane-hydrogel composition can be improved when the composition is formed in the presence of a buffer as compared to a composition that is formed in the substantial absence of a buffer.

### Example 6

### Stability of Lactate Dehydromenase Immobilized in a Polyurethane-Hydrogel Composition

The polyurethane-hydrogel compositions of Examples 4 and 5 were further studied for activity over time to observe the effect of immobilization on enzyme stability.

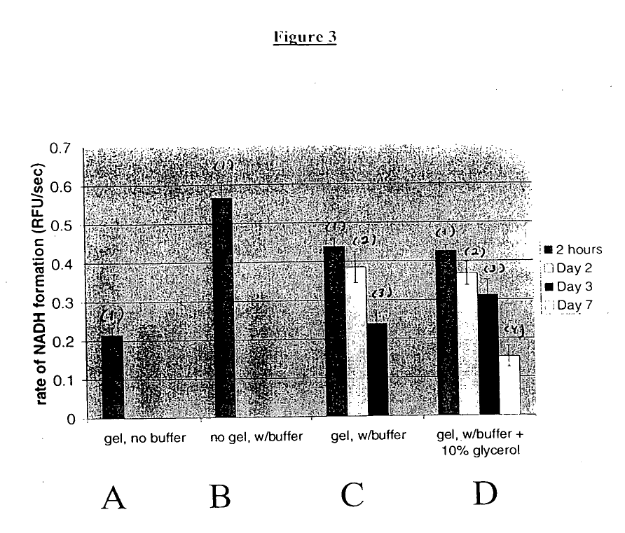

Sample A was prepared according to the procedure for immobilized enzyme in Example 4. Sample B was prepared according to the procedure for free enzyme in Example 5, and Sample C was prepared according to the procedure for immobilized enzyme in Example 5. All samples were stored at room temperature. The enzyme was analyzed for activity as described in Example 3 after 2 hours (1), 48 hours (2), 72 hours (3), and 7 days (4). These results are shown in FIG. 3. Immobilized lactate dehydrogenase (Sample C) retained 54% of its activity after 3 days (3) at room temperature, but free lactate dehydrogenase (Sample B) showed no activity. These results show that immobilized enzyme was more stable over time than the free enzyme.

To determine the effect of an enzyme stabilizer on a polyurethane-hydrogel composition of the invention, this analysis was also conducted on a composition prepared with an enzyme stabilizer (glycerol). Sample D was prepared according to the procedure for immobilized enzyme in Example 5, except that 10% v/v glycerol was added to the biopolymer solution. Sample D still showed 36% activity after 7 days (4) at room temperature (see FIG. 3).

The addition of freshly prepared NAD⁺ to Samples A-D did not increase activity, which may indicate that activity loss was due to enzyme instability not exhaustion of NAD⁺ or degradation of NAD⁺.

### Example 7

### Stability of Lactate Dehydrogenase Immobilized in a Polyurethane-Hydrogel Composition

The effect of an enzyme stabilizer on a polyurethane-hydrogel composition of the invention was further studied by conducting an analysis similar to that of Example 6 with trehalose as an enzyme stabilizer. Four samples were prepared.

Sample A was prepared according to the procedure for immobilized enzyme in Example 5, except that 10% (w/v) trehalose was added to the prepolymer solution.

Sample B was prepared like Sample A, except that 5% (w/v) trehalose was used.

Sample C was free of trehalose and was simply prepared according to the procedure for immobilized enzyme in Example 5.

Sample D was prepared according to the procedure for free enzyme in Example 5, except that 10% (w/v) trehalose was added to the biopolymer solution.

All Samples were stored at room temperature.

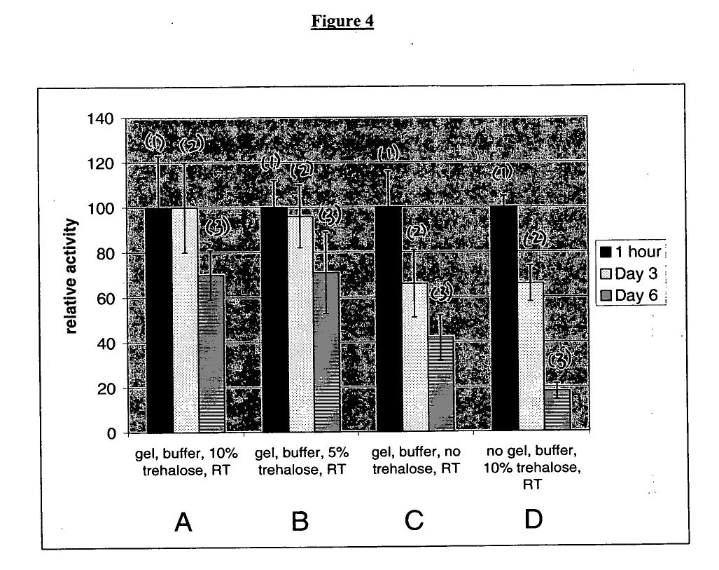

The enzyme was analyzed for activity as described in Example 3 after 1 hour (1), 72 hours (2), and 6 days (3). These results are shown in FIG. 4. Immobilized lactate dehydrogenase prepared with 10% (w/v) trehalose retained 70% activity after 6 days (Sample A) (3), but free lactate dehydrogenase prepared with 10% (w/v) trehalose retained only 18% activity after 6 days (Sample D) (3). These results show that immobilized enzyme was more stable over time than the free enzyme, which indicates that the polyurethane-hydrogel composition enhances stability of an immobilized enzyme as compared to the free enzyme.

### Example 8

### Stability of Lactate Dehydrogenase to Dehydration/Rehydration After Immobilization in a Polyurethane-Hydrogel Composition

To determine the effect of dehydration and rehydration on an enzyme and coenzyme immobilized in a polyurethane-hydrogel composition, lactate dehydrogenase and NAD⁺ were immobilized in a polyurethane-hydrogel composition as described in Example 5. The composition was then dried under N₂ at 30° C. for 3 hours. The dried composition was then rehydrated with H₂O and checked for activity as described in Example 4. The activity was compared to that of free enzyme that was prepared as described in Example 5 and then dried under N₂ at 30° C. for 3 hours. Rehydrated free enzyme showed no activity while rehydrated immobilized enzyme showed 37% activity compared to its activity before dehydration. These results show that an immobilized enzyme was more stable than free enzyme after dehydration.

### Example 9

### Immobilization of β-Galactosidase in a Polyurethane-Hydrogel Composition

To study the stability of yet another enzyme, which catalyzes a different reaction than described in Example 4, the enzyme β-galactosidase (β-Gal) was tested for retention of activity after immobilization in a polyurethane-hydrogel composition.

This enzyme catalyzes the hydrolysis of sugars in the following reaction:
β-D-galactoside (β-gal)+H₂→β-galactose+alcohol.

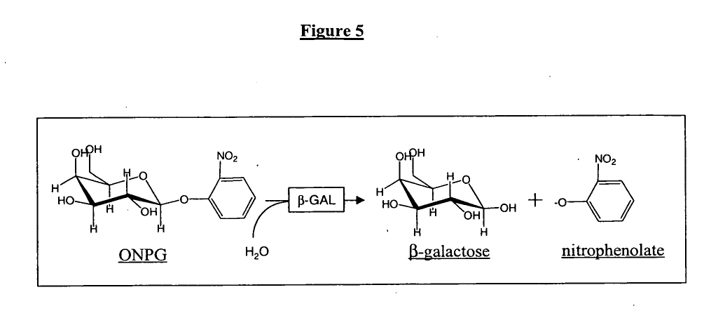

This activity is hydrolytic, while the enzyme in Example 4 has oxidoreductase activity. The β-Gal reaction can be monitored using the chromogenic substrate ONPG (o-nitrophenyl-β-D-galactoside), which yields a bright yellow (λₘₐₓ=420 nm) product nitrophenolate upon hydrolysis. This reaction is shown in FIG. 5.

To immobilize the enzyme in a polyurethane-hydrogel composition, the following steps were taken.

A biopolymer solution having the enzyme, coenzyme, and a crosslinker was prepared. To make this solution, 12 U/ml β-galactosidase (Sigma, Milwaukee, Wis.; catalog number G-5635) and 1% (w/v) low molecular-weight polyethylenimine (Aldrich, Milwaukee, Wis.; catalog number 40,871-9) (polyethylenimine was adjusted to pH=8.0 using 18 M H₂SO₄) were mixed.

A prepolymer solution was also prepared by dissolving through rapid mixing the prepolymer in Example 1 in 50 mM potassium-phosphate buffer at pH 8.0 (pH adjusted with 6 M KOH) to a concentration of 2.5% (w/v).

The prepolymer solution (9 parts) was mixed thoroughly with the biopolymer solution (1 part) within 5 minutes of making the prepolymer solution to form a polymerization mixture. The polymerization mixture was dispensed into a 96-well microtiter plate (300 μl/well). The mixture was left to polymerize for at least 30 minutes at room temperature and standard pressure before the activity was assayed.

The composition was assayed for activity by addition of 0.4 mM ONPG (o-nitrophenyl-β-D-galactoside) (Sigma, catalog number N-1127) in 50 mM potassium phosphate pH 7.0 to each microplate well. The results were obtained by visual inspection. The composition in each well changed from colorless to yellow after ONPG was added. A polyurethane-hydrogel composition that was prepared without immobilizing β-galactosidase was also assayed for activity. No color change was observed upon addition of ONPG. These results show that the enzyme maintained its activity upon immobilization in the composition.

### Example 10

### Immobilization of Enzymes that Participate in a Multienzyme System in a Polyurethane-Hydrogel Composition

To immobilize a multienzyme system in a polyurethane-hydrogel composition of the invention, a multienzyme system was immobilized in the prepolymer of Example 1. Polymerization was initiated concurrently with immobilization, and the polymerization mixture was deposited onto a 96-well microtiter plate (300 μl/well).

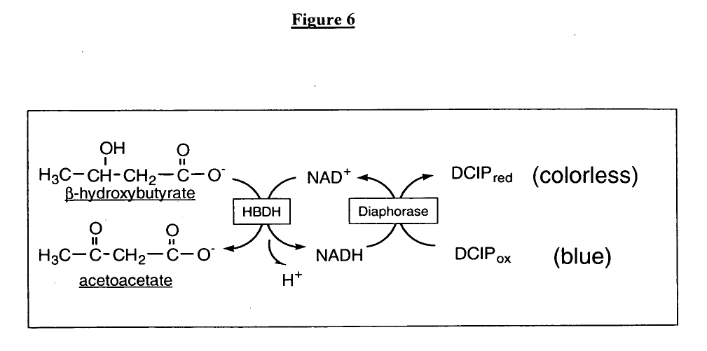

The multienzyme system includes P-hydroxybutyrate dehydrogenase (HBDH), diaphorase, NAD⁺ (nicotinamide adenine dinucleotide), and DCIP (2,6-dichloroindolphenol). This system detects β-hydroxybutyrate by a well-known reaction scheme. According to the reaction scheme, β-hydroxybutyrate dehydrogenase catalyzes the oxidation of β-hydroxybutyrate and the reduction of NAD⁺ to produce acetoacetate and NADH. Diaphorase then catalyzes the transfer of electrons from NADH to DCIP. The catalysis of electron transfer from NADH to DCIP can be monitored by a colorimetric assay because DCIP is dark blue (600 nm) and becomes colorless upon electron reduction. FIG. 6 illustrates this scheme.

To immobilize the enzymes that participate in a multienzyme system in a polyurethane-hydrogel composition, the following steps were taken.

A first biopolymer solution having the multienzyme system and a crosslinker was prepared. To make this solution, a 1% (weight/volume) aqueous solution of low molecular-weight polyethylenimine was prepared as described in Example 2.

8 U/ml β-hydroxybutyrate dehydrogenase (H-9408, Sigma, Milwaukee, Wis.), 4 U/ml diaphorase (D-5540, Sigma), 10 mM NAD⁺ (N-6522, Sigma), and 1 mM DCIP (2,6-dichloroindolphenol; D-1878, Sigma) were then added to the polyethylenimine solution to make the biopolymer solution.

An incomplete multienzyme system was also prepared by omitting β-hydroxybutyrate dehydrogenase from the above mixture. This second biopolymer solution served as an additional control in the experimental analysis.

A prepolymer solution was also prepared according to the procedure in Example 2, except that the prepolymer was dissolved in 50 mM potassium-phosphate buffer having a pH of 8.0 (pH adjusted with 6 M potassium hydroxide) instead of distilled water.

The prepolymer solution (9 parts) was mixed thoroughly with the complete multienzyme system biopolymer solution (1 part) by rapid mixing. 200 μl polymerization mixture were aliquoted into each well of a 96-well microtiter plate. This procedure was repeated using the incomplete multienzyme system biopolymer solution (i.e., the biopolymer solution that lacked β-hydroxybutyrate dehydrogenase). The mixtures were left to polymerize for at least 1 hour at room temperature and standard pressure to form a polyurethane-hydrogel composition. The composition in each well was blue in color by visual inspection.

The composition was then analyzed for activity by a colorimetric assay by adding either 3 mM β-hydroxybutyrate substrate (H-6501, Sigma) or 3 mM NADH (N-8129 in 50 mM potassium phosphate pH 7.0, Sigma) in potassium phosphate pH 7.0 to each well and visually monitoring the composition. Upon addition of β-hydroxybutyrate to wells containing complete multienzyme the polyurethane-hydrogel composition, the color changed from blue to colorless within 10 minutes. This indicates that the multienzyme system was active. When buffer alone (50 mM potassium phosphate at pH 7.0) was added to the composition, no color change was observed. Similarly, no activity was observed when buffer alone or β-hydroxybutyrate was added to wells that contained the incomplete multienzyme system. Addition of NADH to wells containing the incomplete system became colorless within 10 minutes demonstrating that immobilized diaphorase was still functionally active in this composition.

These results show that the enzyme and coenzyme maintained their interplay upon immobilization of the enzymes in the composition, and the enzymes maintained their activity. These results also demonstrate advantages in network permeability and advantages in optical transparency, which are useful for biomedical applications.

### Comparative Example 11

### Effect of Organic Solvent on Activity of Enzyme Immobilized in a Polyurethane-Hydrogel Composition

Organic solvents generally denature or inactivate proteins (see, e.g., Properties and Synthetic Applications of Enzymes in Organic Solvents, ANGEW. CHEM. TNT. Ed., 39: 2226-2254 (2000), and BIOTRANSFORMATIONS IN ORGANIC CHEMISTRY, 2^{nd} Ed., Berlin, Germany: Springer-Verlag (1995), and Stability and Stabilization of Biocatalysts, A. Ballesteros et al. eds. (1998)).

To determine the effect of organic solvent on the activity of an enzyme immobilized in a polyurethane-hydrogel composition, lactate dehydrogenase and NAD⁺ are immobilized in a polyurethane-hydrogel composition as described in Example 5, except that the prepolymer solution contains 30% (w/v) acetonitrile.

After polymerization, the polyurethane-hydrogel composition is washed thoroughly with 50 mM potassium-phosphate buffer having a pH of 8 to remove the acetonitrile from the composition. The enzyme is also analyzed for activity as described in Example 5. Activity of free enzyme is also determined as described in Example 5, except that the solution also includes 30% (w/v) acetonitrile. The immobilized enzyme and the free enzyme both show no activity.

### Example 12

### Use of a Polyurethane-Hydrogel Composition in a Protein Microarray

Five different proteins were immobilized in polyurethane-hydrogel compositions and were arrayed in a microtiter plate to allow the proteins to be assayed in parallel.

Enzymes were immobilized in a polyurethane-hydrogel composition as described in Example 10. For parallel analysis, five different biopolymer solutions were prepared and added to a prepolymer solution to give polymerization mixtures having final concentrations of 1 U/ml diaphorase (Sigma, D-5540), 1 mM NAD+ (Sigma, N-1511 (N-0505 for NADP+ used with glucose-6-phosphate dehydrogenase)), and 1 U/mL of a different dehydrogenase enzyme-glucose-6-phosphate dehydrogenase (Sigma, G-6378) (Sample A), alanine dehydrogenase (Sigma, A-7653) (Sample B), and glutamate dehydrogenase (Sigma, G-2626) (Sample C), lactate dehydrogenase (Sigma, L-1254) (Sample D), β-hydroxybutyrate dehydrogenase (Sigma, H-9408) (Sample E). Each dehydrogenase catalyzes the oxidation of its substrate and the reduction of NAD(P)⁺ to produce NAD(P)H. Diaphorase then catalyzes the transfer of electrons from NAD(P)H to resazurin to form the fluorescent compound resorufin.

Each polyurethane-hydrogel composition was dispensed into a designated location in a 96-well microtiter plate (50 μL/well) and allowed to polymerize for 30 minutes.

The microtiter plate was then assayed by adding 5 mM resazurin (Molecular Probes, R-12204) and a single substrate (3.33 mM glucose-6-phosphate (Sigma, G-7879), 1 mM alanine (Sigma, A-5824), 1 mM glutamate (Sigma, G-2128), 1 mM lactate (Sigma, L-7022), and 1 mM β-hydroxybutyrate substrate (Sigma, H-6501), respectively) to each row and measuring resorufin formation by monitoring fluorescence using a Typhoon 8600 Fluorescence scanner.

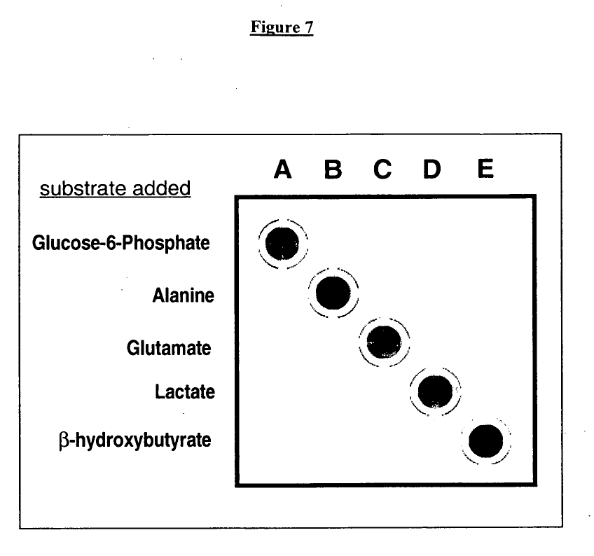

The results of this analysis are shown in FIG. 7. Activity was observed for a specific substrate only in plate wells that contained the complementary dehydrogenase enzyme immobilized in a polyurethane-hydrogel composition. Thus, activity was observed for Sample A with glucose-6-phosphate, Sample B with alanine, Sample C with glutamate, Sample D with lactate, and Sample E with β-hydroxybutyrate.

One skilled in the art will readily understand that robotic control and other high-throughput methods can be used to take advantage of the parallel-assay capability of a polyurethane hydrogel of the invention.

### Example 13

### Use of a Polyurethane-Hydrogel Composition in a Protein Microarray

The protein microarray method described in Example 12 can also be automated.

For parallel analysis, seven different biopolymer solutions are prepared as described in Example 12, and each solution contains 1 U/ml of a different dehydrogenase enzyme—lactate dehydrogenase, β-hydroxybutyrate dehydrogenase, alanine dehydrogenase, glucose dehydrogenase, glutamate dehydrogenase, alcohol dehydrogenase, malate dehydrogenase, and 1 U/ml diaphorase (all available from Sigma).

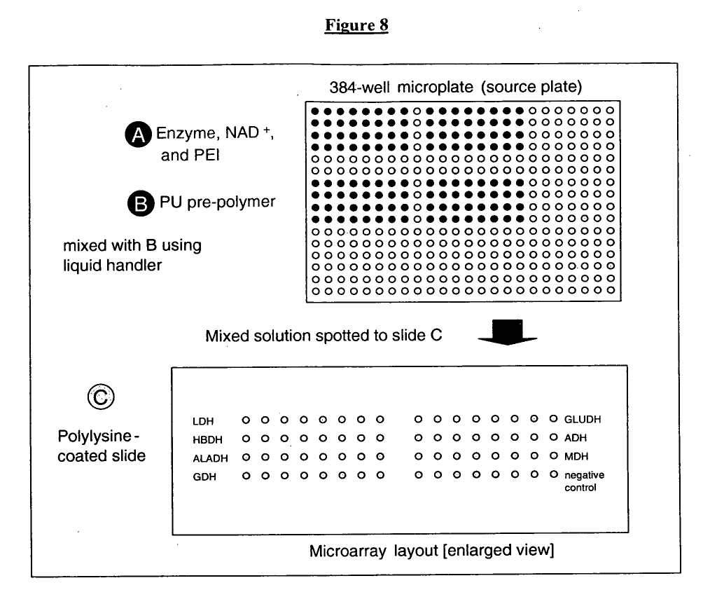

Each polyurethane-hydrogel composition is dispensed onto a microplate using an SDDC-2 microarrayer available from Virtek Vision Corporation (Waterloo, Ontario, Canada). The SDDC-2 is a modular system with a 3-axis robot gantry and a dispenser/pipettor subsystem. FIG. 8 shows a diagram for the automated procedure using the SDCC-2.

In a 384-well microplate, each dehydrogenase enzyme (IOU) is predispensed in replicate (in 8 wells of the plate) in 40 μl of 50 mM potassium-phosphate buffer (pH 8.0) containing 0.1% polyethylenimine, and 1 mM NAD⁺ (shown in A). The 384-well plate also includes an equal number of wells containing predispensed prepolymer solution (1 mg/well) (shown in B). The robot is then programmed to aspirate 8 wells containing enzyme (shown in A)(using a 4-channel electronic pipettor module) to a second set of wells containing the prepolymer (B). The samples are then mixed by repeated aspiration and dispension (5×). This process is rapidly repeated for a set of 4 enzymes (4×8→32 wells). At the completion of this step, a 4×8 pin head spots (˜325 picoliters per spot) the mixed 32 samples onto a poly-L-lysine-coated slide (available from Corning, Cat. # 2549) (shown in C) where the sample is allowed to polymerize. Multiple slides can be made at this step in rapid fashion. The mixing and spotting process is then repeated for the remaining 3 enzymes plus a nonenzyme control.

The slides are assayed in parallel by adding 5 mM resazurin and a single substrate (i.e., immersing slide in 1 mM lactate substrate, β-hydroxybutyrate substrate, etc.) and measuring the rate of resorufin formation by monitoring fluorescence.

The results of this analysis detect activity for a specific substrate at locations that contain a complementary dehydrogenase enzyme immobilized in a polyurethane-hydrogel composition.

### Example 14

### Immobilization of Bacterial Cells in a Polyurethane-Hydrogel Composition

Live bacterial cells were immobilized in a polyurethane-hydrogel composition according to the invention by the following method.

Bacterial cultures were grown according to standard methods known to one of skill in the art. In general, cultures of Escherichia coli (JM109) (available from Promega, Inc., Madison, Wis.) were grown in shake flasks on LB (Luria-Bertani) medium (available from Fisher Scientific, Fairlawn, N.J.). Cultures were harvested during exponential growth by centrifugation (14,000 rpm) and washed twice by resuspension of cell pellets in M9 minimal salts medium followed by centrifugation. After the second wash, cells were resuspended to an OD₆₀₀ of approximately 130-150 in 50 mM potassium-phosphate buffer, pH 8.0 (pH adjusted with 6 M KOH) and placed on ice. These cells provided the live-cell samples. Heat-killed samples were also prepared by incubating washed cells at 100° C. for 20 minutes. The heat-killed cells were then placed on ice.

To immobilize the bacterial cells in a polyurethane-hydrogel composition, a prepolymer solution was mixed with a cell solution within 5 minutes of preparing the prepolymer solution.

The prepolymer solution was prepared by dissolving the prepolymer of Example 1 in 50 mM potassium-phosphate buffer at pH 8.0 to a concentration of 2.5% (w/v).

A live-cell solution was prepared by mixing live bacterial cells, glucose, and low molecular-weight polyethylene imine (Aldrich, Milwaukee, Wis.; catalog number 40,871-9, adjusted to pH=8.0 using 18 M H₂SO₄). The live-cell solution was added to the prepolymer solution to give a polymerization mixture having final concentrations of live cells of OD₆₀₀ ˜1, a final concentration of glucose of 1.5% (w/v), and 0.1% (w/v) of polyethylenimine. The solutions were mixed by inversion and dispensed into a 96-well microtiter plate (300 μl/well). The polymerization mixture was left to polymerize for at least 10 minutes before assaying for cell viability.

A heat killed-cell solution was also prepared and mixed with a prepolymer solution. The heat killed-cell solution was prepared like the live-cell solution, except that heat-killed cells were used in place of the live cells.

The polyurethane-hydrogel composition immobilized with bacterial cells was assayed for cell viability by addition of 0.03 mM PMS (phenazine methosulfate) (Aldrich, catalog number P1,340-1) and 0.16 mM TNBT [(2,2′,5,5′-tetra-p-nitrophenyl-3,3′-[3,3′-dimethoxy-4,4′-diphenyl]ditetrazolium chloride); Sigma, catalog number T-4000] to each of the wells. The results were monitored visually.

PMS and TNBT were used as indicators for actively respiring cells. For this assay, PMS is reduced directly by respiring cells, which then transfers its electrons to TNBT. Upon reduction, TNBT turns from a faint yellow to dark blue (nearly black).

Wells containing immobilized live E. coli cells became dark blue/black within 60 minutes of being exposed to PMS and TNBT, demonstrating that the cells were actively respiring/viable within the polyurethane-hydrogel composition. These results were identical to the positive control, which included live cells that were not immobilized but only suspended in 50 mM potassium-phosphate buffer.

Wells containing immobilized heat-killed E. coli showed no color change, demonstrating that the cells were not viable and that the polyurethane-hydrogel composition did not create a false positive for the live cells. These results were identical to the control, which included heat-killed cells that were not immobilized but only suspended in 50 mM potassium-phosphate buffer.

These assays were repeated approximately 90 minutes after the first assays. The results of these assays were identical to the first set.

### Example 15

### Immobilization of Yeast Cells in a Polyurethane-Hydrogel Composition

Live yeast cells were immobilized in a polyurethane-hydrogel composition according to the invention by the following method.

Yeast cultures are grown according to standard methods known to one of skill in the art. In general, cultures of Saccharomyces cerevisiae (available from Invitrogen, Inc., Carlsbad, Calif.) are grown in shake flasks using YPD medium (available from Clontech, Inc., Palo Alto, Calif.). Cultures are harvested during exponential growth by centrifugation (14,000 rpm) and washed twice by resuspension of cell pellets in M9 minimal salts medium (available from Difco, a division of Becton Dickinson and Co., Sparks, Md.) followed by centrifugation. After the second wash, cells are resuspended to an OD₆₀₀ of approximately 130-150 in 50 mM potassium-phosphate buffer, pH 8.0 (pH adjusted with 6 M KOH) and placed on ice. These cells provided the live-cell samples. Heat-killed samples are also prepared by incubating washed cells at 100° C. for 20 minutes. The heat-killed cells are then placed on ice.

To immobilize the yeast cells in a polyurethane-hydrogel composition, a prepolymer solution is mixed with a cell solution within 5 minutes of preparing the prepolymer solution.

The prepolymer solution is prepared by dissolving the prepolymer of Example 1 in 50 mM potassium-phosphate buffer at pH 8.0 to a concentration of 2.5% (w/v).

A live-cell solution is prepared by mixing live yeast cells, glucose, and low molecular-weight polyethylenimine (Aldrich, Milwaukee, Wis.; catalog number 40,871-9, adjusted to pH=8.0 using 18 M H₂SO₄). The live-cell solution is added to the prepolymer solution to give a polymerization mixture having final concentrations of live cells of OD₆₀₀˜1, a final concentration of glucose of 1.5% (w/v), and 0.1% (w/v) of polyethylenimine. The solutions are mixed by inversion and dispensed into a 96-well microtiter plate (300 μl/well). The polymerization mixture is left to polymerize for at least 10 min before assaying for cell viability.

A heat killed-cell solution is also prepared and mixed with a prepolymer solution. The heat killed-cell solution is prepared like the live-cell solution, except that heat-killed cells are used in place of the live cells.

The polyurethane-hydrogel composition immobilized with yeast cells is assayed for cell viability by using a commercially available yeast cell viability kit (Molecular Probes, Inc., Eugene, Oreg., catalog number L-7009). This kit contains a two-color fluorescent probe that exploits endogenous biochemical processing mechanisms. Live cells exhibit orange-red fluorescence, while dead cells exhibit bright diffuse green-yellow fluorescence.

Wells containing immobilized live S. cerevisiae cells exhibit orange-red fluorescence demonstrating that the cells are viable within the polyurethane-hydrogel composition. These results are identical to the positive control, which included live cells that are not immobilized but only suspended in 50 mM potassium-phosphate buffer.

Wells containing immobilized heat-killed S. cerevisiae exhibit green-yellow fluorescence demonstrating that the cells are not viable and that the polyurethane-hydrogel composition does not create a false positive for the live cells. These results are identical to the control, which included heat-killed cells that are not immobilized but only suspended in 50 mM potassium-phosphate buffer.

These assays are repeated approximately 90 minutes after the first assays. The results of these assays are identical to the first set.

### Example 16

### Immobilization of Mammalian Cells in a Polyurethane-Hydrogel Composition

Live mammalian cells are immobilized in a polyurethane-hydrogel composition according to the invention by the following method.

Mammalian cell cultures are grown according to standard methods known to one of skill in the art. In general, cultures of CHO (Chinese Hamster Ovary cells) (available from ATCC, catalog number CCL-61) are grown in Ham's F12K medium with 2 mM L-glutamine adjusted to contain 1.5 g/L sodium bicarbonate, 90%; fetal bovine serum, 10% (available from ATCC, catalog number 30-2004) at 37° C. Cultures are harvested during exponential growth and washed with PBS medium according to Sambrook and Russell in Molecular Cloning: A Laboratory Manual (2000). After the second wash, cells are resuspended to an OD₆₀₀ of approximately 130-150 in 50 mM potassium-phosphate buffer, pH 8.0 (pH adjusted with 6 M KOH) and placed on ice. These cells provided the live-cell samples. Heat-killed samples are also prepared by incubating washed cells at 80° C. for 5 minutes. The heat-killed cells are then placed on ice.

To immobilize the mammalian cells in a polyurethane-hydrogel composition, a prepolymer solution is mixed with a cell solution within 5 minutes of preparing the prepolymer solution.

The prepolymer solution is prepared by dissolving the prepolymer of Example 1 in 50 mM potassium-phosphate buffer at pH 8.0 to a concentration of 2.5% (w/v).

A live-cell solution is prepared by mixing live mammalian cells, glucose, and low molecular-weight polyethylenimine (Aldrich, Milwaukee, Wis.; catalog number 40,871-9, adjusted to pH=8.0 using 18 M H₂SO₄). The live-cell solution is added to the prepolymer solution to give a polymerization mixture having final concentrations of live cells of OD₆₀₀ ˜1, a final concentration of glucose of 1.5% (w/v), and 0.1% (w/v) of polyethylenimine. The solutions are mixed by inversion and dispensed into a 96-well microtiter plate (300 μl/well). The polymerization mixture is left to polymerize for at least 10 minutes before assaying for cell viability.

A heat killed-cell solution is also prepared and mixed with a prepolymer solution. The heat killed-cell solution is prepared like the live-cell solution, except that heat-killed cells are used in place of the live cells.

The polyurethane-hydrogel composition immobilized with mammalian cells is assayed for cell viability by using a commercially available cell viability kit (Molecular Probes, Inc., Eugene, Oreg., catalog number L-3224). This kit contains a two-color fluorescent probe that exploits endogenous biochemical processing mechanisms. Live cells exhibit green fluorescence, while dead cells exhibit red fluorescence.

Wells containing immobilized live CHO mammalian cells exhibit green fluorescence demonstrating that the cells are viable within the polyurethane-hydrogel composition. These results are identical to the positive control, which included live cells that are not immobilized but only suspended in 50 mM potassium-phosphate buffer.

Wells containing immobilized heat-killed CHO mammalian cells exhibit red fluorescence demonstrating that the cells are not viable and that the polyurethane-hydrogel composition does not create a false positive for the live cells. These results are identical to the control, which included heat-killed cells that are not immobilized but only suspended in 50 mM potassium-phosphate buffer.

These assays are repeated approximately 90 minutes after the first assays. The results of these assays are identical to the first set.

### Example 17

### Immobilization of Bacterial Cells Expressing Recombinant DNA in a Polyurethane-Hydrogel Composition

Bacterial cells expressing DNA that encodes for a specific protein are immobilized in a polyurethane-hydrogel composition. The DNA transformation and gene expression was done by a known method as described in Technical Bulletin No. 095 entitled E. coli Competant Cells provided by Promega Corporation.

Commercially available competent cell cultures of E. coli JM109 strain (Promega product number L2001) were transformed with pGEM-3Z vector (available from Promega and provided with product number L2001) as described in protocol for the E. coli Competant Cells. Briefly, frozen competant cells were thawed on ice and 100 μl transferred to a pre-chilled culture tube. The cells were then transformed by adding 1-50 ng pGEM-3Z expression vector and heat shocking. The cells were placed on ice and 900 μl of 4° C. SOC medium added to the transformation reaction. The reaction was then incubated for 60 minutes at 37° C. with shaking. After transformation, the cells were diluted 1:10 and 100 μl plated on LB/ampicillin plates.

The pGEM-3Z vector contains a gene that encodes for β-galactosidase controlled by a specific promoter (lac) and also contains the selection marker gene Amp^{r} (ampicillin resistance). The transformed cells were then used to inoculate two shake flasks (25 ml media) containing nutrient broth with ampicillin. The inducer IPTG (Promega catalog number V3955) was added (1 mM) to only one of the shake flasks. The flasks were incubated overnight at 37° C. with shaking. The cells were harvested, washed and resuspended in phosphate buffer as described in Example 14. It should be noted that the DNA transformation and protein expression as described in this Example should not be limited to this specific gene, DNA vector, or bacterial host. Transformation and gene expression for any particular gene or protein of interest can be performed by known methods such as those described in Sambrook and Russell eds., Molecular Cloning: A Laboratory Manual (2000).

To immobilize the bacterial cells in a polyurethane-hydrogel composition, a prepolymer solution was mixed with each cell solution (i.e., induced cells and noninduced cells) within 5 minutes of preparing the prepolymer solution.

The prepolymer solution was prepared by dissolving the prepolymer of Example 1 in 50 mM potassium-phosphate buffer at pH 8.0 to a concentration of 2.5% (w/v).

An induced bacterial cell solution was prepared by mixing induced bacterial cells, glucose, and low molecular-weight polyethylenimine (Aldrich, Milwaukee, Wis.; catalog number 40,871-9, adjusted to pH=8.0 using 18 M H₂SO₄). The induced bacterial cell solution was added to the prepolymer solution to give a polymerization mixture having final concentrations of induced cells of OD₆₀₀˜1, a final concentration of glucose of 1.5% (w/v), and 0.1% (w/v) of polyethylenimine. A second noninduced bacterial cell solution was prepared as described above. The solutions were mixed by inversion and dispensed into a 96-well microtiter plate (100 μl/well). The polymerization mixtures were left to polymerize for at least 10 minutes.

In vivo detection of β-galactosidase expression was done by adding 33 μM of the fluorescent ≢2-galactosidase substrate analog C₁₂FDG (available form Molecular Probes, catalog number I-2904) and monitored using a microplate reader with excitation at 497 nm and emission at 518 nm.

The results show that only cells that are induced (i.e., IPTG added) have expressed β-galactosidase activity and that this activity was detectable after cells were immobilized in the polyurethane-hydrogel composition. Low activity was observed in the control experiment where cells were not induced with IPTG. These results show that transformed bacterial cells expressing proteins can be immobilized in a polyurethane-hydrogel composition and retain their activity after immobilization. This composition is particularly useful in the context of cell arrays where several genes of interest can be transformed, expressed, immobilized in a polyurethane hydrogel, and assayed for activity in parallel.

One skilled in the art will recognize that experiments similar to those described in this Example are also useful for other cells including yeast and mammalian cells.

### Comparative Example 18

### Effect of Organic Solvent on Bacterial Cells Immobilized in a Polyurethane-Hydrogel Composition

To determine the effect of organic solvent on bacterial cells immobilized in a polyurethane-hydrogel composition, live E. coli cells are immobilized in a polyurethane-hydrogel composition as described in Example 14, except that the prepolymer solution contained 30% (w/v) acetonitrile. After polymerization, the polyurethane-hydrogel composition is washed thoroughly with 50 mM potassium-phosphate buffer having a pH of 8.0 to remove the acetonitrile from the composition.

The bacterial cells are analyzed as described in Example 14. The results are comparable to those described for heat-killed cells in Example 14.

### Example 19

### Protein-Ligand Interaction in a Polyurethane-Hydrogel Composition

To study the binding ability of a protein that is immobilized in a polyurethane-hydrogel composition, the protein avidin was immobilized in a polyurethane-hydrogel composition and tested for its ability to bind biotinylated β-galactosidase.

Avidin is a biotin-binding protein that is used in a wide variety of applications for detection or purification of biotinylated macromolecules. Biotinylated-β-galactosidase is β-galactosidase modified by chemical attachment of biotin. Biotinylated-β-galactosidase catalyzes the identical reaction described in Example 9.

To immobilize avidin in a polyurethane-hydrogel composition, the following steps were taken.

The prepolymer solution was prepared by dissolving the prepolymer of Example 1 in water to a concentration of 2.5% (w/v).

Biopolymer solutions were prepared by mixing buffer and low molecular-weight polyethylenimine (Aldrich, Milwaukee, Wis.; catalog number 40,871-9, adjusted to pH =8.0 using 12 M HCl) with varying concentrations of avidin (NeutrAvidin, Pierce Chemical Company, Rockford, Ill., catalog number 31000). The biopolymer solutions were added individually to the prepolymer solution to give polymerization mixtures having final concentrations of avidin of 0 (Sample A), 0.0125 (Sample B), 0.025 (Sample C), 0.05 (Sample D), and 0.1 (Sample E) % (w/v), respectively, and polyethylenimine (0.015% (w/v)) in 20 mM potassium-phosphate buffer, pH 8.0 (pH adjusted with 6 M KOH). The solutions were mixed by inversion and spotted (10 μl/spot), in duplicate, onto a glass microscope slide. The polymerization mixtures were left to polymerize for at least 30 minutes and then rinsed with potassium-phosphate buffer for 10 minutes.

The composition was assayed for binding activity as follows. The spotted slide was treated with (0.2 U/ml) biotinylated β-galactosidase (Pierce Chemical, 29939) by immersing the slide in 20 mM potassium-phosphate buffer, pH 8.0 for 90 minutes with gentle agitation. The slide was washed with 100 mM potassium-phosphate buffer, pH 8.0, and the activity of bound biotinylated β-galactosidase was assayed by immersing the slide in assay buffer (100 mM sodium phosphate, pH 8.0 containing 10 mM KCl, 1 mM MgSO₄, and 330 mM β-mercaptoethanol) and adding 10 μM of the fluorescent substrate analog C₁₂FDG (Molecular Probes, 1-2904) for about 10 minutes. The hydrolyzed fluorescent product formed from the β-galactosidase reaction was then measured by fluorescence scanning with a Typhoon 8600 Fluorescence scanner.

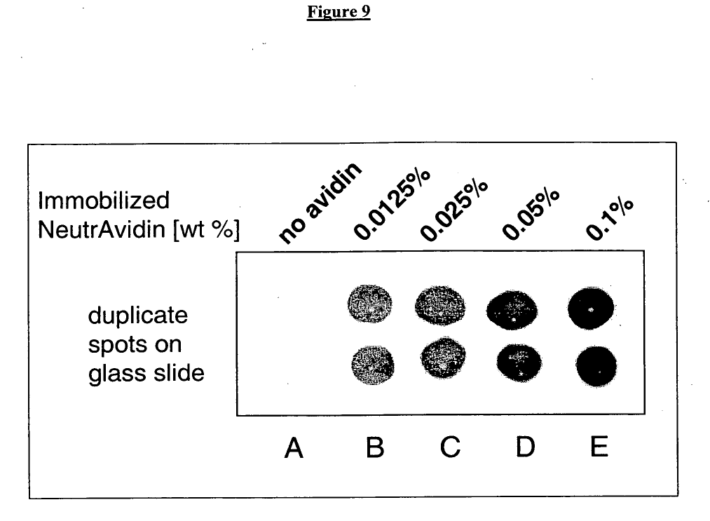

The results from this analysis are shown in FIG. 9. The polymerization mixtures containing immobilized avidin showed relative fluorescence signals that increased with higher concentrations of immobilized avidin (Samples B-E). This demonstrates that the immobilized avidin retained its biotin-binding activity and that the biotinylated β-galactosidase-avidin complex maintained its activity within the polyurethane-hydrogel composition. No fluorescence was observed in spots without immobilized avidin, which indicates minimal nonspecific binding of biotinylated P-galactosidase to the polyurethane-hydrogel composition (Sample A).

### Example 20

### An Immunoassay in a Polyurethane-Hydrogel Composition

To study the ability of an antibody to bind to an antigen that is immobilized in a polyurethane-hydrogel composition, Samples similar to those described in Example 19 were prepared and then treated with fluorescently-labeled antibody (Ab) that cross-reacts with biotin. Thus, avidin was immobilized in a polyurethane-hydrogel composition and treated with biotinylated β-galactosidase as described in Example 19 and then assayed for its ability to react with an antibiotin antibody.

The composition was assayed for the ability of antibiotin Ab to cross-react with avidin-bound, biotinylated-β-galactosidase as follows. A spotted slide containing varying amounts of immobilized avidin (prepared according to Example 19) was treated with (0.2 U/ml) biotinylated β-galactosidase (Pierce Chemical, 29939) by immersing the slide in PBS, pH 8.0 for 60 minutes with gentle agitation. The slide was washed with PBS buffer and then treated with 1:400 diluted antibiotin Ab (50 μl of commercial stock into 20 ml) labeled with Cy3 (Sigma, C-5585) for 2 hours. Bound antibiotin Ab was then measured by fluorescence scanning with a Typhoon 8600 Fluorescence scanner.

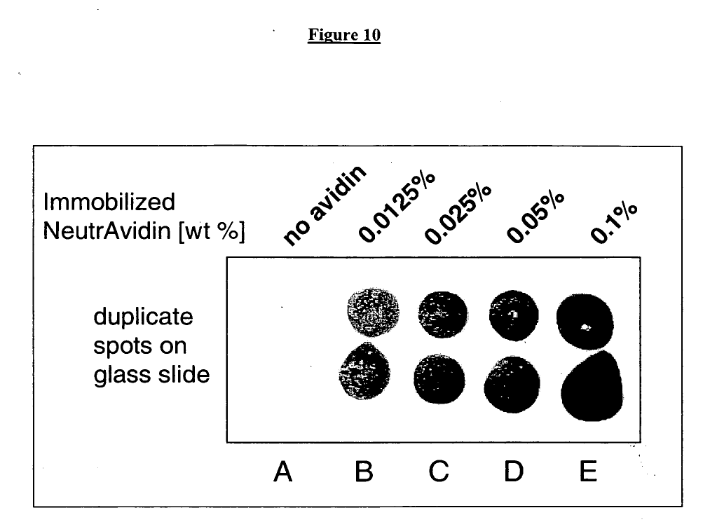

The results from this analysis are shown in FIG. 10. The fluorescent signal due the binding of the labeled antibiotin Ab directly corresponded to the amount of biotinylated-β-galactosidase:avidin complex immobilized in the polyurethane-hydrogel composition (Samples B-E). No fluorescence was observed in spots without immobilized avidin, which indicates very low nonspecific binding of labeled antibiotin Ab to the polyurethane-hydrogel composition (Sample A). These results demonstrate that the polyurethane-hydrogel composition does not interfere with the immunogenic binding activity of an antibody to its target antigen.

### Example 21

### DNA Hydridization in a Polyurethane-Hydrogel Composition

An amine 5′-end-capped probe oligonucleotide was immobilized in a polyurethane composition and tested for its ability to hybridize to a fluorescently labeled target oligonucleotide containing a complementary nucleotide sequence.

To immobilize DNA in a polyurethane-hydrogel composition, the following steps were taken.

The prepolymer solution was prepared by dissolving the prepolymer of Example 1 in water to a concentration of 2.5% (w/v).

Biopolymer solutions were prepared by mixing buffer and low molecular-weight polyethylenimine (Aldrich, Milwaukee, Wis.; catalog number 40,871-9, adjusted to pH=8.0 using 12 M HCl) with varying concentrations of amine 5′-end-capped oligonucleotide (Synthesized and purchased from Operon Technologies, Inc., Alameda, Calif.).

The oligonucleotide contained the following 50 base-pair nucleotide sequence:

| | |
|---|---|
| 5′-CCATTATTAGGTGATGGTATTTTTACTTTGGATGGTGAAGGTTGGAAACA-3′. | (SEQ ID NO: 1) |

The biopolymer solutions were added individually to the prepolymer solution to give polymerization mixtures having final concentrations of oligonucleotide of 0 (Sample A), 80 (Sample B), 40 (Sample C), 20 (Sample D), and 10 (Sample E) nM, respectively, and polyethylenimine (0.015 % (w/v)) in 20 mM potassium-phosphate buffer, pH 8.0 (pH adjusted with 6 M KOH). The solutions were mixed by inversion and spotted (2 μl/spot) onto a clean glass microscope slide (Catalog number SMC-25 available from ArrayIt, Sunnyvale, Calif.). The polymerization mixtures were left to polymerize for at least 30 minutes and then blocked with 20 ml succinic anhydride (17 mg/ml) in 43 mM borate buffer (mixture adjusted to pH 8.2 using 5 M NaOH) for 5 minutes at room temperature. After blocking, the slide was washed three times briefly with potassium-phosphate buffer, pH 8.0.

The composition was assayed for DNA hybridization as follows. Each spot on the slide was treated with 20 μl of 80 nM Cy3-labeled DNA (synthesized and purchased from Operon Technologies, Inc.) complementary to the immobilized probe DNA and allowed to incubate for 90 minutes at room temperature in the dark. The slide was then washed in phosphate buffer overnight (16 hours). Hybridization of labeled target DNA was measured by fluorescence scanning with a Typhoon 8600 Fluorescence.

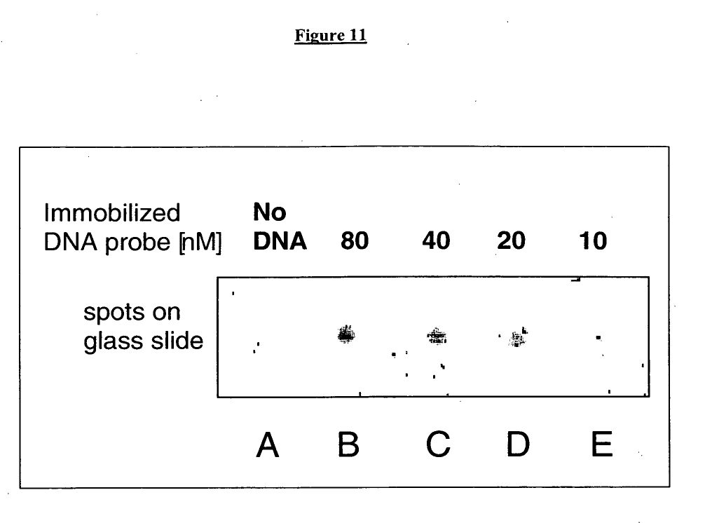

The hybridization results are shown in FIG. 11. The polyurethane-hydrogel compositions containing immobilized DNA showed relative fluorescence signals that increased as the higher concentration of immobilized DNA increased (Samples B-E). Some fluorescence was observed in the spot without immobilized probe DNA, which indicates some, although low, nonspecific binding of labeled target DNA to the polyurethane-hydrogel composition (Sample A).

### Example 22

### Protein-DNA Interaction in a Polyurethane-Hydrogel Composition

To study the binding ability of a protein that is immobilized in a polyurethane-hydrogel composition, the transcription factor NFκB was immobilized in a polyurethane-hydrogel composition and tested for its ability to bind to a fluorescently labeled target oligonucleotide containing the NFκB consensus target nucleotide sequence.

To immobilize NFκB in a polyurethane-hydrogel composition, the following steps were taken.

The prepolymer solution was prepared by dissolving the prepolymer of Example 1 in water to a concentration of 2.5% (w/v).

Biopolymer solutions were prepared by mixing buffer and low molecular-weight polyethylenimine (Aldrich, Milwaukee, Wis.; catalog number 40,871-9, adjusted to pH=8.0 using 12 M HCl) with varying amounts of NFκB (p50) (Promega, Madison, Wis., catalog nuber E3770). The biopolymer solutions were added individually to the prepolymer solution to give polymerization mixtures having final concentrations of NFκB of 0 (Sample A), 0.015 (Sample B), 0.03 (Sample C), 0.06 (Sample D), 0.125 (Sample E), 0.25 (Sample F), 0.5 (Sample G), 1 (Sample H), and 2 (Sample I) U/ml, respectively, and polyethylenimine (0.015% (w/v)) in 20 mM potassium-phosphate buffer, pH 8.0 (pH adjusted with 6 M KOH). The solutions were mixed by inversion and spotted (10 μl/spot), onto a clean glass microscope slide (ArrayIt, SMC-25). The polymerization mixtures were left to polymerize for 30 minutes.

The composition was assayed for DNA binding as follows. The oligonucleotide used was 5′-labeled with fluorescein and contained the consensus target site sequence for NFκB binding site(purchased from Integrated DNA Technologies, Inc., Coralville, Iowa).

| | |
|---|---|
| 5′-TCT GAG GGA CTT TCC TGA TC-3′. | (SEQ ID NO: 2) |

This oligonucleotide was annealed to its complementary strand, and the slide was incubated with 20 nM of the double-stranded fluorescein-labeled target DNA in PBS buffer overnight at room temperature. The slide was then washed in 20 ml potassium-phosphate buffer three times. Binding of labeled target DNA was measured by fluorescence scanning with a Typhoon 8600 Fluorescence scanner.

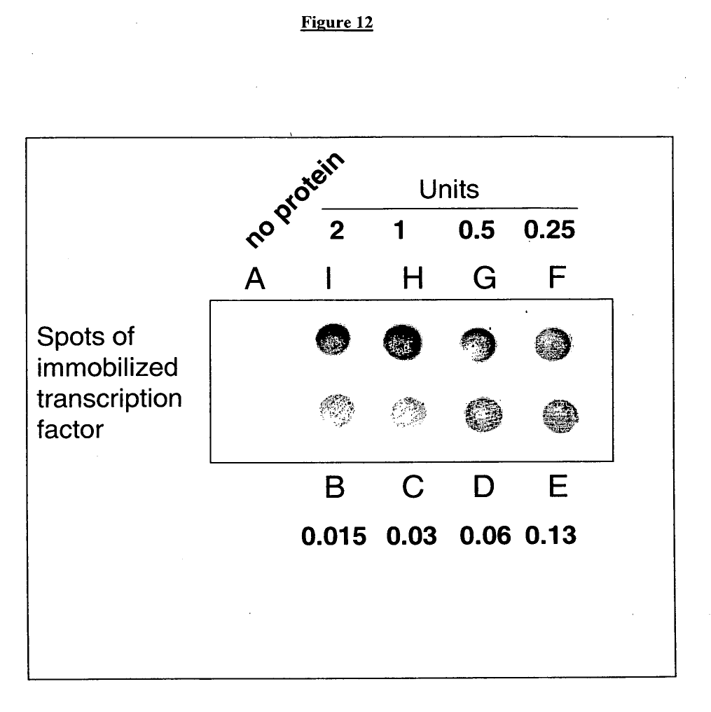

The results are shown in FIG. 12. The polymerization mixtures containing immobilized NFκB showed relative fluorescence signals that increased as concentrations of NFκB increased (Samples B-I). Low relative fluorescence was observed in the spot without immobilized NFκB, which indicates some, although low, nonspecific binding of labeled target DNA to the polyurethane-hydrogel composition (Sample A).

### Example 23

### Immobilization of a Protein in a Polyurethane-Hydrogel Composition After Polymerization

To immobilize a protein in a polyurethane-hydrogel composition after polymerization, the homobifunctional molecule glutaraldehyde (Aldrich, catalog number G400-4) was tested as an immobilizing agent. Polymerization of the polyurethane-hydrogel composition was performed first and then a fluorescently-labeled fibrinogen (Molecular Probes, catalog number F-13191) was immobilized in the polyurethane-hydrogel composition after polymerization using glutaraldehyde.

To immobilize fibrinogen in a polyurethane-hydrogel composition after polymerization, the following steps were taken.

The prepolymer solution was prepared by dissolving the prepolymer of Example 1 in water to a concentration of 2.5% (w/v).

Buffer and low molecular-weight polyethylenimine (Aldrich, Milwaukee, Wis.; catalog number 40,871-9, adjusted to pH=8.0 using 12 M HCl) were added individually to the prepolymer solution to give a polymerization mixture having a final concentration of polyethylenimine (0.1% (w/v)) in 20 mM potassium-phosphate buffer, pH 8.0 (pH adjusted with 6 M KOH). The solution was mixed by inversion and spotted (10 μl/spot) in triplicate onto two clean glass microscope slides (ArrayIt, SMC-25). The polymerization mixtures were left to polymerize for 30 minutes.

After polymerization was complete, one slide (A) was immersed in PBS (pH 8.0), the second slide (B) was immersed in PBS containing glutaraldehyde (5% (v/v)), and both were incubated for 3 hours with gentle agitation. The slides were rinsed briefly with PBS and then each slide was incubated in fresh solutions (as above) in the presence of 40 nM fluorescently-labeled fibrinogen for 90 minutes. The slides were then washed in PBS for 16 hours. Binding of labeled fibrinogen was measured by fluorescence scanning with a Typhoon 8600 Fluorescence scanner.

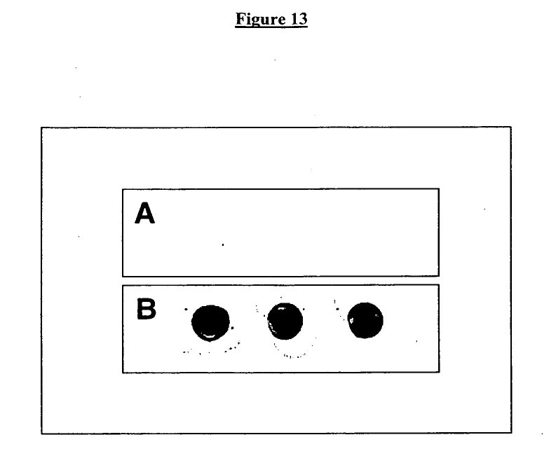

As shown in FIG. 13, no binding of labeled fibrinogen was detected for the slide that did not include the glutaraldehyde treatment (slide A). But the slide that was treated with glutaraldehyde shows an intense signal due to the binding of fluorescently-tagged fibrinogen (slide B). It should be noted that no intrinsic fluorescence was observed for a polyurethane-hydrogel composition solely treated with glutaraldehyde (no addition of labeled protein), which indicates that the observed fluorescence was due to the binding of labeled protein alone.

### Example 24

### Immobilization of a Protein in a Polyurethane-Hydrogel Composition After Polymerization

To immobilize a protein in a polyurethane-hydrogel composition after polymerization, the homobifunctional molecule sulfo-ethylene glycol bis(succinimidylsuccinate) (sulfo-EGS) (Pierce, catalog number 21566) was tested as an immobilizing agent. Polymerization of the polyurethane-hydrogel composition was performed first and then a fluorescently-labeled fibrinogen (Molecular Probes, catalog number F-13191) was immobilized in the polyurethane-hydrogel composition after polymerization using sulfo-EGS.

To immobilize fibrinogen in a polyurethane-hydrogel composition after polymerization, the following steps were taken.

The prepolymer solution was prepared by dissolving the prepolymer of Example 1 in water to a concentration of 2.5% (w/v).

Buffer and low molecular-weight polyethylenimine (Aldrich, Milwaukee, Wis.; catalog number 40,871-9, adjusted to pH=8.0 using 12 M HCl) were added individually to the prepolymer solution to give a polymerization mixture having a final concentration of polyethylenimine (0.1% (w/v)) in 20 mM potassium-phosphate buffer, pH 8.0 (pH adjusted with 6 M KOH). The solution was mixed by inversion, and spotted (10 μl/spot) in quadruplicate onto two clean glass microscope slides (ArrayIt, SMC-25). The polymerization mixtures were left to polymerize for 30 minutes. After polymerization was complete, the slides were washed in PBS, pH 8.0. One slide (A) was then immersed in PBS containing 40 nM fluorescently-labeled fibrinogen, and the second slide (B) was then immersed in PBS containing 40 nM fluorescently-labeled fibrinogen with 5 mM sulfo-EGS. Both slides were incubated for 2 hours with gentle agitation. The slides were then washed extensively in PBS for 24 hours. Binding of labeled fibrinogen was measured by fluorescence scanning with a Typhoon 8600 Fluorescence scanner.

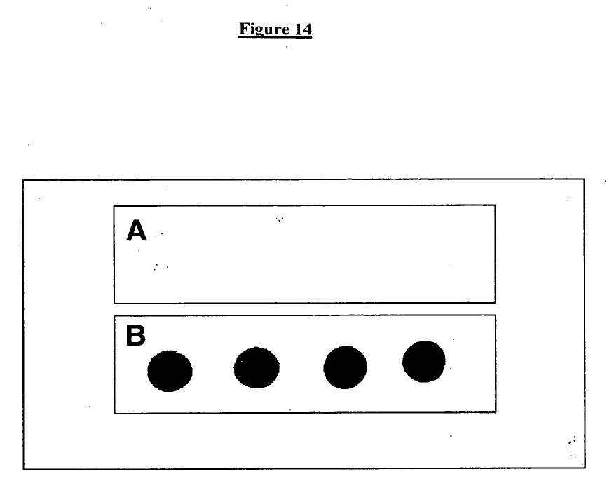

As shown in FIG. 14, no binding of labeled fibrinogen was detected for the slide that did not include the sulfo-EGS treatment (slide A). But the slide that was treated with sulfo-EGS shows an intense signal due to the binding of fluorescently-tagged fibrinogen (slide B). It should be noted that no intrinsic fluorescence was observed for a polyurethane-hydrogel composition treated with sulfo-EGS alone (no addition of labeled protein, which indicates that the observed fluorescence was due to the binding of labeled protein alone).

### Example 25

### Immobilization of a Multicomponent Enzyme System in a Polyurethane-Hydrogel Composition

To immobilize a multicomponent enzyme system in a polyurethane-hydrogel composition of the invention, a multicomponent enzyme system was immobilized in the prepolymer of Example 1. Polymerization was initiated concurrently with immobilization, and the polymerization mixture was deposited onto a 96-well microtiter plate.

The multicomponent enzyme system included an NADPH P450 reductase enzyme component and a cytochrome P450 monooxygenase (CYP1A2) enzyme component. According to the reaction scheme, the reductase component transfers electrons from NADPH to the P450 monooxygenase component. The reduced monooxygenase is then able to catalyze the dealkylation of methoxyresorufin to yield the fluorescent product resorufin. Glucose-6-phosphate and glucose-6-phosphate dehydrogenase were added to regenerate NADPH during the course of the reaction. FIG. 15 illustrates this scheme.

To immobilize the multicomponent enzyme system in a polyurethane-hydrogel composition, the following steps were taken.

The prepolymer solution was prepared by dissolving the prepolymer of Example 1 in water to a concentration of 2.5% (w/v).

A biopolymer solution was prepared by mixing buffer, low molecular-weight polyethylenimine (Aldrich, Milwaukee, Wis.; catalog number 40,871-9, adjusted to pH=8.0 using 12 M HCl), cytochrome P450 monooxygenase enzyme mix (Pan Vera, Madison, Wis., catalog number P2304), glucose-6-phosphate, and glucose-6-phosphate dehydrogenase.

The biopolymer solution was added to the prepolymer solution to give a polymerization mixture having final concentrations of polyethylenimine (0.015% (w/v)), cytochrome P450 monooxygenase (25 nM), glucose-6-phosphate (3.33 mM), and glucose-6-phosphate dehydrogenase (0.4 U/ml) in 40 mM potassium-phosphate buffer, pH 8.0 (pH adjusted with 6 M KOH).

The polymerization mixture was thoroughly mixed, and 100 μl of the mixture were aliquoted into each well of a 96-well microtiter plate. The mixture was left to polymerize for at least 1 hour at room temperature and standard pressure to form a polyurethane-hydrogel composition.

A control sample was also prepared by mixing the prepolymer solution described above with potassium-phosphate buffer, pH 8.0 (final concentration of 40 mM) with polyethylenimine (final concentration of 0.015% (w/v)).

The multicomponent enzyme system and the control sample were analyzed for activity at various time points at room temperature by adding 5 mM methoxyresorufin substrate (Molecular Probes) and 1 mM NADPH (N-1630 in 50 mM potassium-phosphate buffer, pH 7.0, Sigma) to each well (final reaction volume equal to 200 μl) and then incubating for 1 day or 5 days depending on the sample. The amount of resorufin product formed was then monitored using a SpectraMax GeminiXS microplate fluorescence reader (excitation=λ₁ 532 nm; emission=λ₂ 580 nm). This analysis was carried out according to the manufacturer's instructions.

The control sample showed about 0.4 rfu after 1 day and about 0.3 rfu after 5 days. In contrast, the sample having the multienzyme system showed about 6.3 rfu after 1 day and about 5.2 rfu after 5 days. The enzyme system substantially maintained its activity after incubation for 5 days at room temperature (showing a 17% decrease in activity). These results show that the individual components of the enzyme system maintained their activity and interplay upon immobilization in the polyurethane-hydrogel composition. This activity was maintained over multiple days, which indicates that the hydrogel composition is substantially stable.

### Example 26

### Polyurethane-Hydrogel Compositions Having Reduced Nonspecific Protein Binding

In certain polyurethane-hydrogel applications, it may be desirable to reduce or optimize, and perhaps minimize, nonspecific protein binding to a polyurethane hydrogel. To investigate a composition that reduces nonspecific protein binding, varying concentrations and types of water-soluble crosslinkers were tested. More particularly, a lower concentration of polyethylenimine was used as the amine crosslinker (as compared to the other Examples provided in this specification), as well as alternative crosslinkers with lower amine functionality than polyethylenimine. A substantial amount of fluorescently-labeled protein was introduced to these polyurethane-hydrogel compositions, and the polyurethane hydrogels were tested for nonspecific binding of the labeled proteins.

To investigate polyurethane-hydrogel compositions that exhibit reduced nonspecific protein binding as compared to polyurethane-hydrogel compositions having 0.1% (w/v) of polyethylenimine, the following steps were taken.

The prepolymer solution was prepared by dissolving the prepolymer of Example 1 in water to a concentration of 2.5% (w/v).

Buffer and low molecular-weight polyethylenimine (Aldrich, Milwaukee, Wis.; catalog number 40,871-9) were added individually to the prepolymer solution to give a polymerization mixture having a final concentration of polyethylenimine (0.1% (w/v)) in 20 mM potassium-phosphate buffer, pH 8.0 (Sample A). A second mixture was prepared similar to Sample A, except that the polyethylenimine final concentration was reduced to 0.015% (w/v) (Sample B). A third mixture was prepared similar to the first two, except polyethylenimine was substituted with a 3-arm amine end-capped polyethyleneglycol (catalog number 0J2V0L13, available from Shearwater Corporation, Huntsville, Ala.) to a final concentration of 0.8% (w/v) (Sample C). A fourth mixture was prepared similar to the first two, except polyethylenimine was substituted with a polyoxyethylene bis(amine) (Sigma, catalog number P-9906) (Sample D) to a final concentration of 0.8% (w/v). The solution was mixed by inversion and spotted (10 μl/spot), in duplicate, onto two clean glass microscope slides (Arraylt, SMC-25). The polymerization mixtures were left to polymerize for 120 minutes.

After polymerization, the slides were washed with PBS, pH 8.0 for 10 minutes. To one slide, each spot was overlayed with 20 μl of 1 mg/ml fluorescein-labeled BSA (bovine serum albumin) (Molecular Probes, A-23015) so that the spots were completely covered by the labeled protein. Each of the spots on the second slide were overlayed with 20 μl of 1 mg/ml fluorescein-labeled ovalbumin (Molecular Probes, O-23020). The slides were then incubated at room temperature in the dark for 2 hours. The slides were washed in PBS, pH 8.0 for 48 hours. Nonspecific binding of the fluorescein-labeled proteins was measured by fluorescence scanning with a Typhoon 8600 Fluorescence scanner.

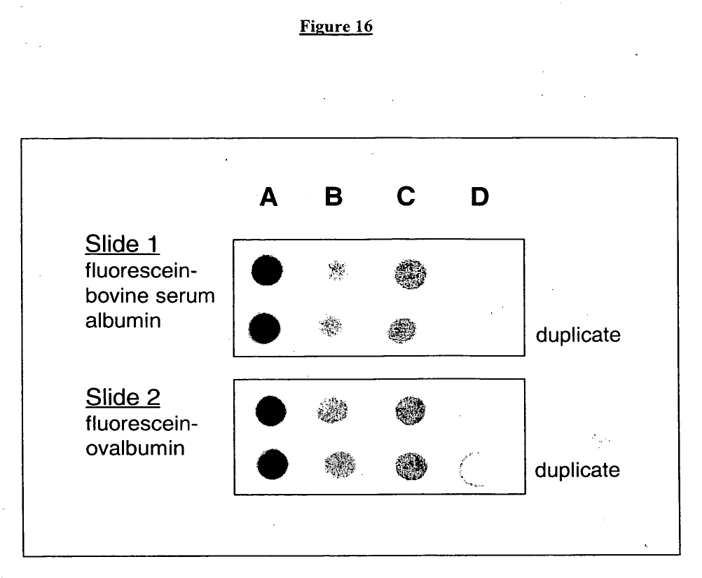

The polyurethane hydrogel prepared from 0.1% (w/v) polyethylenimine (Sample A) showed the greatest amount of nonspecific protein binding relative to all compositions tested. When the amount of polyethylenimine was reduced to 0.015% (w/v) (Sample B), less nonspecific binding occurred relative to the polyurethane hydrogel prepared from 0.1% (w/v). Moreover, when the water-soluble crosslinker was changed such that the number of available amine groups on the crosslinker was reduced (i.e., changed to a 3-arm amine end-capped polyethyleneglycol (Sample C) or polyoxyethylene bis(amine) (Sample D)), less nonspecific binding occurred relative to the polyurethane hydrogel prepared from 0.1% (w/v). These results are shown in FIG. 16.

### Example 27

### Polyurethane-Hydrogel Compositions Having Reduced Nonspecific Protein Binding

To investigate polyurethane-hydrogel compositions that exhibit reduced nonspecific protein binding as compared to polyurethane-hydrogel compositions having 0.1% (w/v) of polyethylenimine, the following steps were taken.

Bacterial cellular lysate was prepared as follows. E. coli JM109 cells (Promega, catalog number L2001) were grown overnight at 37° C. with shaking in a 250-mL beveled flask containing 75 mL LB. The cells were harvested by centrifugation at 6,000 rpm for 40 minutes at 4° C. The broth was decanted, and the cell pellet was resuspended in 7.5 mL of 20 mM potassium-phosphate buffer, pH 8.0. The cells were lysed by sonication, and the insoluble cell debris was removed by centrifugation at 12,000 rpm for 1 hour. The supernatant was then used as the source of protein for the investigation.

The prepolymer solution was prepared by dissolving the prepolymer of Example 1 in water to a concentration of 2.5% (w/v).

Buffer and low molecular-weight polyethylenimine (Aldrich, Milwaukee, Wis.; catalog number 40,871-9) were added individually to the prepolymer solution to give a polymerization mixture having a final concentration of polyethylenimine (0.1% (w/v)) in 20 mM potassium-phosphate buffer, pH 8.0 (Sample A). A second mixture was prepared similar to Sample A, except that the polyethylenimine final concentration was reduced to 0.015% (w/v) (Sample B). A third mixture was prepared similar to the first two, except polyethylenimine was substituted with a 3-arm amine end-capped polyethyleneglycol (catalog number 0J2V0L13, available from Shearwater Corporation, Huntsville, Ala.) to a final concentration of 0.8% (w/v) (Sample C). A fourth mixture was prepared similar to the first two, except polyethylenimine was substituted with a polyoxyethylene bis(amine) (Sigma, catalog number P-9906) (Sample D) to a final concentration of 0.8% (w/v). The solution was mixed by inversion and spotted (10 μl/spot), in duplicate, onto two clean glass microscope slides (Arraylt, SMC-25). The polymerization mixtures were left to polymerize for 120 minutes.

After polymerization, the slides were washed with potassium-phosphate buffer, pH 8.0 for 10 minutes. One slide was overlayed with cell lysate so that the spots were completely covered by the crude mixture of proteins and allowed to incubate for 2 hours. The second slide was used as a nontreated control. The slides were then washed three times with potassium-phosphate buffer, pH 8.0 and then soaked in 0.05% fresh SDS (sodium dodecyl sulfate) (Sigma) for 30 minutes. The slides were then soaked in Sypro Red (Molecular Probes, catalog number S-6653) staining solution at 1× concentration in 7.5% acetic acid for 16 hours. The slides were washed in 7.5% acetic acid for 1 minute and then imaged with a Typhoon 8600 Fluorescence scanner for nonspecific binding of proteins. Intensity of spots were quantified by subtracting the fluorescence intensities (relative fluorescence units) of corresponding spots from the nontreated control slide.

The polyurethane hydrogel prepared from 0.1% (w/v) polyethylenimine (Sample A) showed the greatest amount of nonspecific protein binding relative to all compositions tested. When the amount of polyethylenimine was reduced to 0.015% (w/v) (Sample B), less nonspecific binding occurred relative to the polyurethane hydrogel prepared from 0.1% (w/v). Moreover, when the water-soluble crosslinker was changed such that the number of available amine groups on the crosslinker was reduced (i.e., changed to a 3-arm amine end-capped polyethyleneglycol (Sample C) or polyoxyethylene bis(amine) (Sample D)), less nonspecific binding occurred relative to the polyurethane hydrogel prepared from 0.1% (w/v).

Sample A showed nonspecific binding of nearly 100,000 rfu, but all other Samples showed substantially less. Sample B showed nonspecific binding of about 35,000 rfu, and Samples C and D showed nonspecific binding of about 40,000 rfu. Thus, the nonspecific binding was reduced by about 60% of that shown for Sample A.

### Example 28

### Chemical Treatment of a Polyurethane Hydrogel to Reduce Nonspecific Protein Binding

To investigate a method that reduces nonspecific protein binding to a polyurethane hydrogel prepared from 0.1% (w/v) polyethylenimine, a polyurethane-hydrogel composition was treated postpolymerization using acetic anhydride. A substantial amount of fluorescently-labeled protein was introduced to the treated polyurethane hydrogel, and the polyurethane hydrogel was tested for nonspecific binding of the labeled protein.

To determine whether the polyurethane-hydrogel composition that was treated with acetic anhydride exhibits reduced nonspecific protein binding relative to a polyurethane hydrogel prepared from 0.1% (w/v) polyethylenimine, the following steps were taken.

The prepolymer solution was prepared by dissolving the prepolymer of Example 1 in water to a concentration of 2.5% (w/v).

Buffer and low molecular-weight polyethylenimine (Aldrich, Milwaukee, Wis.; catalog number 40,871-9) were added individually to the prepolymer solution to give a polymerization mixture having a final concentration of polyethylenimine (0.1% (w/v)) in 20 mM potassium-phosphate buffer, pH 8.0. The solution was mixed by inversion and spotted (10 μl/spot), in triplicate, onto two clean glass microscope slides (Arraylt, SMC-25). The polymerization mixtures were left to polymerize for 30 min.

After polymerization, one slide was immersed in phosphate buffered saline (PBS), pH 8.0 (Slide A). The other slide (Slide B) was immediately treated with 43 mM borate, pH 8.3 containing 17 mg/mL acetic anhydride (Aldrich, catalog number 11,004-3) and 2% acetic acid (mixture adjusted to pH 8.2 with 6 M KOH) for 10 minutes. Slide B was then washed three times with PBS, pH 8.0 for 5 minutes. Both slides A and B were then treated with fluorescently-labeled fibrinogen (Molecular Probes, catalog number F-13191) in PBS, pH 8.0, for 2 hours. Both slides were washed three times with PBS, pH 8.0 for 30 minutes each wash. Binding of the fluorescently-labeled fibrinogen was measured by fluorescence scanning with a Typhoon 8600 Fluorescence scanner.

Slide A showed nonspecific binding with an average of 6656 rfu, but Slide B showed substantially less with an average of 3186 rfu. Thus, the nonspecific binding was reduced by about 52% of that shown for the composition on Slide A.

### Example 29

### Stability of Glucose-6-Phosphate Dehydrogenase Immobilized in a Polyurethane-Hydrogel Composition

The effect of an enzyme stabilizer on a polyurethane-hydrogel composition of the invention was further studied by conducting an analysis similar to that for Example 7, except glucose-6-phosphate dehydrogenase was used as enzyme instead of lactate dehydrogenase.

First, glucose-6-posphate dehydrogenase was immobilized in a polyurethane-hydrogel composition as described in Example 12, except that diaphorase was not coimmobilized in the polyurethane-hydrogel composition. The samples were stored at 4° C. for 24 hours or 5 days depending on the sample.

Second, a control sample was prepared according to the procedure for free enzyme described in Example 5.

The sample having immobilized glucose-6-phosphate dehydrogenase (immobilized sample) and the control sample were analyzed for activity by adding 1 U/ml diaphorase, 3.3 mM glucose-6-phosphate, and 5 mM resazurin. Resorufin product formation was measured by fluorescent scanning with a Typhoon 8600 Fluorecence scanner. The results showed that the immobilized sample had activity of about 41,000 rfu after 24 hours and still had about 97% of this activity after 5 days. In contrast, the control sample had activity of about 40,000 rfu after 24 hours and only had about 3.5% of this activity after 5 days. These results show that immobilized enzyme was more stable over time than the free enzyme, which indicates that the polyurethane-hydrogel composition enhances stability of an immobilized enzyme as compared to the free enzyme.

### Example 30

### Chemical Treatment of the Polyethylenimine Crosslinker Prior to Polyurethane Hydrogel Polymerization to Reduce Nonspecific Protein Binding

To investigate a method that reduces nonspecific protein binding to a polyurethane hydrogel prepared from 0.1% (w/v) polyethylenimine (PEI), the PEI is first treated with glycidol prior to use as a crosslinker in a polyurethane-hydrogel composition. A substantial amount of fluorescently-labeled protein is introduced to the polyurethane hydrogel containing glycidol-modified PEI (as the crosslinker), and this modified polyurethane hydrogel composition tested for nonspecific binding of the labeled protein.

To determine whether the glycidol-modified PEI polyurethane-hydrogel composition exhibits reduced nonspecific protein binding relative to a polyurethane hydrogel prepared from unmodified 0.1% (w/v) polyethylenimine, the following steps are taken.

Ten ml of 20% (w/v) low molecular-weight polyethylenimine (Aldrich; catalog number 40,871-9) is added to 10 ml of 40% (w/v) glycidol (Aldrich, catalog number G580-9) in water adjusted to pH 10.0 with 12 M HCl and allowed to incubate for 2 h at room temperature. After incubation, the mixture is adjusted to pH 8.0 using 12 M HCl.

The prepolymer solution is prepared by dissolving the prepolymer of Example 1 in water to a concentration of 2.5% (w/v).

Buffer and low molecular-weight polyethylenimine (Aldrich, Milwaukee, Wis.; catalog number 40,871-9) are added individually to the prepolymer solution to give a polymerization mixture having a final concentration of polyethylenimine (0.1% (w/v)) in 20 mM potassium-phosphate buffer, pH 8.0. A second solution is prepared by adding buffer and glycidol-modified polyethylenimine to the prepolymer solution to give a polymerization mixture having a final concentration of glycidol-modified polyethylenimine (0.1% (w/v)) in 20 mM potassium-phosphate buffer, pH 8.0. The solutions are mixed by inversion and spotted (10 μl/spot), in triplicate, onto a clean glass microscope slides (ArrayIt, SMC-25) (Slides A and B, respectively). The polymerization mixtures are left to polymerize for 30 min.

After polymerization, both slides are washed with phosphate buffered saline (PBS), pH 8.0. Both slides A and B are then treated with fluorescently-labeled fibrinogen (Molecular Probes, catalog number F-13191) in PBS, pH 8.0, for 2 hours. Both slides are washed three times with PBS, pH 8.0 for 30 min each wash. Binding of the fluorescently-labeled fibrinogen is measured by fluorescence scanning with a Typhoon 8600 Fluorescence scanner.

Slide A shows higher fluorescence relative to Slide B indicating that nonspecific protein binding is reduced by glycidol modification of the polyethylenimine crosslinker prior to its use in the polyurethane hydrogel composition.

1. A polyurethane-hydrogel composition having an immobilized biologic, said composition being prepared by a method comprising:
(a) admixing at least one prepolymer and at least one water-soluble amine-functionalized crosslinker in aqueous solvent and in the substantial absence of organic solvent, said prepolymer being prepared from at least one water-soluble polyol and at least one isocyanate to form a polyurethane-hydrogel mixture; and
(b) contacting said mixture with a biologic to immobilize the biologic in said mixture to form a composition having an immobilized biologic,
wherein said composition is transparent, is substantially polymerized, and has an effective number-average molecular weight between crosslinks.

2. A composition according to claim 1, wherein contacting said mixture with a biologic comprises derivatizing at least one of said prepolymer and said crosslinker with said biologic before admixing said prepolymer said crosslinker.

3. A composition according to claim 1, wherein contacting said mixture with a biologic comprises admixing said biologic with said prepolymer and said crosslinker.

4. A composition according to claim 1, wherein said mixture is polymerized before contacting said mixture with said biologic.

5. A composition according to claim 1, wherein at least one of said mixture and said biologic is contacted with an immobilizing agent either before or concurrently with contacting said mixture with said biologic.

6. A composition according to claim 1, wherein said crosslinker comprises polyethylenimine.

7. A composition according to claim 1, wherein said crosslinker comprises an amine end-capped poly(ethylene oxide) crosslinker.

8. A composition according to claim 1, wherein said crosslinker comprises at least one of a 3-arm amine end-capped polyethyleneglycol and polyoxyethylene bis(amine).

9. A composition according to claim 1, wherein said crosslinker comprises a polyamine, said polyamine having a charge density of at least 0.8 meq charge per gram of crosslinker.

10. A composition according to claim 1, wherein said crosslinker has a functionality effective to provide a reaction rate with said prepolymer that is at least 10 times faster than the reaction rate of water with said prepolymer.

11. A composition according to claim 1, wherein said crosslinker is selected to optimize nonspecific binding to said composition.

12. A composition according to claim 1, wherein said prepolymer is added in an amount of no greater than 5 weight percent, said weight percent being based on the total weight of all components.

13. A composition according to claim 1, wherein said prepolymer is prepared from an aliphatic or cycloaliphatic isocyanate.

14. A composition according to claim 13, wherein said isocyanate comprises isophorone diisocyanate.

15. A composition according to claim 1, wherein said prepolymer is prepared from a polyoxyalkylene polyol.

16. A composition according to claim 15, wherein said polyol comprises a 7,000 molecular-weight triol copolymer of 75% ethylene oxide and 25% propylene oxide.

17. A composition according to claim 1, wherein said prepolymer is prepared from an isocyanate comprising isophorone diisocyanate and a polyol comprising a 7,000 molecular-weight triol copolymer of 75% ethylene oxide and 25% propylene oxide.

18. A composition according to claim 1, wherein said biologic comprises a cell.

19. A composition according to claim 1, wherein said biologic comprises a peptide.

20. A composition according to claim 19, wherein said peptide comprises a protein.

21. A composition according to claim 20, wherein said protein comprises an enzyme.

22. A composition according to claim 1, wherein said biologic comprises a nucleic, a peptide nucleic acid, or a combination of a nucleic and a peptide nucleic acid.

23. A composition according to claim 1, wherein said biologic comprises a saccharide.

24. A composition according to claim 1, wherein said admixing step further comprises admixing at least one additional hydrogel component with said at least one prepolymer and at least one crosslinker.

25. A biomedical device suitable for immobilizing a biologic, said device comprising:
(a) a substrate; and
(a) a polyurethane-hydrogel composition suitable for immobilizing a biologic, said composition being adhered to said substrate and said composition being prepared by a method comprising:
(i) admixing at least one prepolymer and at least one water-soluble amine-functionalized crosslinker in aqueous solvent and in the substantial absence of organic solvent to form a polyurethane-hydrogel mixture, said prepolymer being prepared from at least one water-soluble polyol and at least one isocyanate; and
(ii) polymerizing said mixture to form a composition suitable for immobilizing a biologic,
wherein said composition is transparent, is substantially polymerized, and has an effective number-average molecular weight between crosslinks.

26. A biomedical device according to claim 25, wherein said composition is prepared from a prepolymer comprising an aliphatic or cycloaliphatic isocyanate.

27. A biomedical device according to claim 26, wherein said isocyanate comprises isophorone diisocyanate.

28. A biomedical device according to claim 25, wherein said prepolymer is prepared from a polyoxyalkylene polyol.

29. A biomedical device according to claim 28, wherein said polyol comprises a 7,000 molecular-weight triol copolymer of 75% ethylene oxide and 25% propylene oxide.

30. A biomedical device according to claim 25, wherein said prepolymer is prepared from an isocyanate comprising isophorone diisocyanate and a polyol comprising a 7,000 molecular-weight triol copolymer of 75% ethylene oxide and 25% propylene oxide.

31. A biomedical device according to claim 25, said composition being prepared from a crosslinker being selected to provide reduced nonspecific binding to said composition as compared to a device having a composition being prepared from 0.1 weight-percent polyethylenimine, said weight percent being based on total weight of all components.

32. A biomedical device according to claim 31, wherein said crosslinker comprises an amine end-capped poly(ethylene oxide) crosslinker.

33. A biomedical device according to claim 31, wherein said crosslinker comprises at least one of a 3-arm amine end-capped polyethyleneglycol and polyoxyethylene bis(amine).

34. A biomedical device according to claim 25, wherein a biologic is immobilized in said composition and said composition has an immobilized biologic, said biologic being immobilized in said composition by contacting said mixture with said biologic and the contacting step occurring before, concurrently with, or after the polymerizing step.

35. A biomedical device according to claim 34, wherein at least one of said mixture and said biologic is contacted with an immobilizing agent either before or concurrently with contacting said mixture with said biologic.

36. A biomedical device according to claim 34, wherein said biologic comprises at least one of a cell, a peptide, a nucleic acid, a peptide nucleic acid, and a saccharide.

37. A biomedical device according to claim 25, said device being suitable for conducting an assay that identifies lead compounds, assesses protein function, identifies protein-protein interactions, identifies protein substrates, or identifies protein-small molecule interactions.

38. A biomedical device according to claim 25, wherein said device provides high-throughput analysis of a probe sample.

39. A biomedical device according to claim 25, wherein said device is included in a kit, said kit comprising at least one reagent useful for conducting an assay on said device.

40. A biomedical device according to claim 25, wherein said device comprises at least one of a test strip, a protein array, a DNA array, a DNA microarray, a cell array, and a cell microarray.

41. A method for preparing a transparent polyurethane-hydrogel composition having an immobilized biologic:
(a) admixing at least one prepolymer and at least one water-soluble amine-functionalized crosslinker in aqueous solvent and in the substantial absence of organic solvent to form a polyurethane-hydrogel mixture, said prepolymer prepared from at least one water-soluble polyol and at least one isocyanate; and
(b) contacting said mixture with a biologic to immobilize the biologic in said mixture to form a composition having an immobilized biologic,
wherein said composition is transparent, is substantially polymerized, and has an effective number-average molecular weight between crosslinks.

42. A method according to claim 41, wherein contacting said mixture with a biologic comprises derivatizing at least one of said prepolymer and said crosslinker with said biologic before admixing said prepolymer with said crosslinker.

43. A method according to claim 41, wherein contacting said mixture with a biologic comprises admixing said biologic with said prepolymer and said crosslinker.

44. A method according to claim 41, wherein said mixture is polymerized before contacting said mixture with said biologic.

45. A method according to claim 41, wherein at least one of said mixture and said biologic is contacted with an immobilizing agent either before or concurrently with contacting said mixture with said biologic.

46. A method according to claim 41, wherein said crosslinker comprises polyethylenimine.

47. A method according to claim 41, wherein said crosslinker comprises an amine end-capped poly(ethylene oxide) crosslinker.

48. A method according to claim 41, wherein said crosslinker comprises at least one of a 3-arm amine end-capped polyethyleneglycol and polyoxyethylene bis(amine).

49. A method according to claim 41, wherein said crosslinker comprises a polyamine, said polyamine having a charge density of at least 0.8 meq charge per gram of crosslinker.

50. A method according to claim 41, wherein said crosslinker has a functionality effective to provide a reaction rate with said prepolymer that is at least 10 times faster than the reaction rate of water with said prepolymer.

51. A composition according to claim 41, wherein said crosslinker is selected to optimize nonspecific binding to said composition.

52. A composition according to claim 41, wherein said prepolymer is added in an amount of no greater than 5 weight percent, said weight percent being based on the total weight of all components.

53. A method according to claim 41, wherein said prepolymer is prepared from an aliphatic or cycloaliphatic isocyanate.

54. A method according to claim 41, wherein said isocyanate comprises isophorone diisocyanate.

55. A method according to claim 41, wherein said prepolymer is prepared from a polyoxyalkylene polyol.

56. A method according to claim 55, wherein said polyol comprises a 7,000 molecular-weight triol copolymer of 75% ethylene oxide and 25% propylene oxide.

57. A method according to claim 41, wherein said prepolymer is prepared from an isocyanate comprising isophorone diisocyanate and a polyol comprising a 7,000 molecular-weight triol copolymer of 75% ethylene oxide and 25% propylene oxide.

58. A method according to claim 41, wherein said biologic comprises at least one of a cell, a peptide, a nucleic acid, a peptide nucleic acid, and a saccharide.

59. A method of conducting a biomedical assay, said method comprising:
(a) obtaining a polyurethane-hydrogel composition having an immobilized biologic, said composition being prepared by a method comprising:
(i) admixing a prepolymer and a water-soluble amine-functionalized crosslinker in aqueous solvent and in the substantial absence of organic solvent to form a polyurethane-hydrogel mixture, said prepolymer being prepared from at least one water-soluble polyol and at least one isocyanate;
(ii) contacting said mixture with a biologic to immobilize the biologic in said mixture; and
(iii) polymerizing said mixture, said polymerized mixture being transparent, being substantially polymerized, and having an effective number-average molecular weight of crosslinks;
(b) contacting said composition with a probe sample; and
(c) detecting interaction between said probe sample and said biologic.

60. A method according to claim 59, wherein, for step (a), the contacting step (ii) and the polymerizing step (iii) can be carried out stepwise or concurrently.

61. A method according to claim 60, wherein the contacting step (ii) occurs after the polymerizing step (iii).

62. A method according to claim 59, wherein said crosslinker comprises polyethylenimine.

63. A method according to claim 59, wherein said crosslinker comprises an amine end-capped poly(ethylene oxide) crosslinker.

64. A method according to claim 59, wherein said crosslinker comprises at least one of a 3-arm amine end-capped polyethyleneglycol and polyoxyethylene bis(amine).

65. A method according to claim 59, wherein said crosslinker is selected to optimize nonspecific binding to said composition.

66. A method according to claim 59, wherein said prepolymer is prepared from an isocyanate comprising isophorone diisocyanate and a polyol comprising a 7,000 molecular-weight triol copolymer of 75% ethylene oxide and 25% propylene oxide.

67. A method according to claim 59, wherein said biologic comprises at least one of a cell, a peptide, a nucleic acid, a peptide nucleic acid, and a saccharide.
